(19) United States
(12) Patent Application Publication
(10) Pub. No.: US 2004/0102525 A1
(43) Pub. Date: May 27, 2004

(54) Compositions and methods of treating neurological disease and providing neuroprotection

(76) Inventor: Walter E. Kozachuk, Kensington, MD (US)

Correspondence Address: Thomas P. Liniak, Liniak, Berenato & White, Suite 240, 6550 Rock Spring Drive, Bethesda, MD, 20817, US

(21) Appl. No.: 10/442,985

(22) Filed: May 22, 2003

Related U.S. Application Data
(60) Provisional application No. 60/382,072, filed on May 22, 2002.

(51) Int. Cl. A61K 31/13

(52) U.S. Cl. 514/662

(57) ABSTRACT

Pharmaceutical compositions and methods of use thereof for the acute, chronic and prophylactic treatment of neurologic and neurodegenerative diseases, attenuation of acute or chronic neuronal damage in neurological disease (“neuroprotection”), and prophylaxis of neurological diseases, where the neurological diseases may involve excessive stimulation of the NMDA receptor, hypofunction of the NMDA receptor, up- or down regulation of the NMDA receptor, and abnormal subunit structure or function of the NMDA receptor. The pharmaceutical compositions are open-channel antagonists of the NMDA (N-methyl-D-aspartate) receptor complex, and include memantine (a 1-amino-3,5-dimethyl-adamantane hydrochloride), felbamate, acamprosate, and MRZ 2/579. The invention relates to oral, controlled or sustained release, intravenous, rectal, transcutaneous or other preparations such as lipid emulsion or crystal technology.

### FIELD OF INVENTION

The present invention relates to pharmaceutical compositions which are open-channel antagonists of the NMDA (N-methyl-D-aspartate) receptor complex, specifically memantine (a 1-amino-3,5-dimethyl-adamantane hydrochloride). Memantine is hypothesized to bind at the Mg++ site or multiple other sites in the channel of the NMDA receptor. The invention relates to methods of use for the acute, chronic and prophylactic treatment of neurologic and neurodegenerative diseases, attenuation of acute or chronic neuronal damage in neurological disease (“neuroprotection”), and prophylaxis of neurological diseases. Neurological diseases may involve excessive stimulation of the NMDA receptor, hypofunction of the NMDA receptor, up- or down regulation of the NMDA receptor, and abnormal subunit structure or function of the NMDA receptor. The invention relates to oral, controlled or sustained release, intravenous, rectal, transcutaneous or other preparations such as lipid emulsion or crystal technology. The trade name is Akatinol Memantine® (Merz and Co.) The invention also relates to the compounds felbamate, acamprosate, and MRZ 2/579.

### BACKGROUND OF THE INVENTION

L-glutamate is the major excitatory neurotransmitter in the central nervous system and acts on the NMDA receptor. The ionotropic NMDA receptor, which fluxes both calcium and sodium, is located on the neuronal cell surface and has multiple binding sites (i.e., glycine, polyamine, NMDA) as well as an ion-channel which has several internal binding sites (i.e., Mg++, PCP). The NMDA receptor has important functions in learning and memory, apoptosis, neuronal migration-development-differentiation, synaptogenesis, and the regulation of developmental cell death. Unique properties of this receptor include: voltage-dependency, a high permeability to Ca++, a requirement for coactivation by glycine, and blockade by physiological concentrations of Mg++. These features are responsible for its specific role in the fundamental basis of learning or LTP (long-term potentiation), the process where strong excitatory stimulation causes potentiation of subsequent stimuli along the same pathway as well as LTD (long-term depression). Glutamate has also been implicated in the pathogenesis of numerous acute and chronic neurological disorders by multiple mechanisms.

Mechanism of Action

Memantine is classified as an open-channel NMDA blocker. Memantine may act at the Mg++ site or multiple other sites in the channel of the NMDA receptor. The mechanism of action of the uncompetitive NMDA receptor antagonist memantine is similar to the potent Mg++ ion. Mg++ is an endogenous low affinity channel blocker with rapid kinetics and is required for NMDA receptor-dependent function such as synaptic plasticity. Memantine blocks and unblocks the open NMDA receptor channels with double exponential kinetics: the amplitude and speed of the fast component of the block increases with memantine concentration, while the speed of fast unblock remains constant but the amplitude decreases with memantine concentration. Memantine does not completely block all of the NMDA receptors, leaving 20% of the channels unblocked, which are thus available for subsequent physiological activation. This property allows blockage of tonic low level NMDA receptor activity but unblocking during relevant synaptic activation. At physiological conditions, both Mg++ and memantine occupy the NMDA receptor channel and both exit the NMDA receptor channel after synaptic depolarization due to their voltage-blocking dependency and rapid unblocking kinetics. With prolonged depolarization, memantine leaves the channel less easily as Mg++ and therefore, therapeutic concentrations of memantine provide more protective against the neurotoxic effects of NMDA receptor agonists. At low Mg++ concentrations, maximum voltage-dependent blockade of NMDA channels occurs in the presence of memantine. Thus, memantine is a potent surrogate for Mg++ and prevents excessive calcium entry into the neuron by binding to the Mg++ as well as other sites at the NMDA channel. These antagonistic effects of memantine at the NMDA receptor were not reversed by glycine concentrations suggesting no interaction at the strychnine-insensitive glycine modulatory site at the NMDA receptor-channel complex. Memantine is also a weak antagonist at the L- and N type voltage-activated calcium channels, Na+ channels, but has no effect on GABA or AMPA receptors.

Memantine has multiple mechanisms of action that are hypothesized to produce its safety and efficacy profile. These include at least: (1) use-dependent channel blocking or the binding and blocking of agonist gated open channels more rapidly than closed channels, (2) low binding affinity or faster effective blocking rates, (3) rapid intrinsic association kinetics, (4) rapid dissociation kinetics and voltage-dependency which allow blocking during synaptic depolarization but allows physiologic neuronal activity, (5) NMDA subunit selectivity in which memantine preferentially blocks the NR2C and NR2D subunits and to a lesser degree the NR2B subunit, (6) partial trapping or the mechanism where a fraction of the blocker can escape from the closed channel, (7) multiple actions at the NMDA receptor or allosteric non-competitive actions, (8) and actions at other receptor targets. The moderate sensitivity of memantine at the NR2B receptor is an important function, since both NMDA mediated LTP and LTD were abolished in NR2B knock-out mice. Thus, the above properties produce less behavioral toxicity and may account for the reduced side effects and favorable adverse event profile. In addition, transient NMDA receptor inactivation has been shown to provide long-term protection and decreased apoptosis in cultured cortical neurons from multiple death signals. The transient inactivation appears to trigger a rapid compensatory survival response against both apoptotic and non-apoptotic cell death mechanisms. Thus, these latter results imply efficacy for prophylaxis in chronic neurological diseases.

In pathological states, NMDA receptors are activated acutely by higher concentrations of glutamate or by chronic sub-acute concentrations of glutamate. Under these conditions, Mg++ leaves the NMDA channel upon moderate depolarization but the blocking kinetics, degree of voltage dependency, and rapid dissociation from the NMDA channel make therapeutic levels of memantine more effective than Mg++ in protection against neurotoxicity. Thus, efficacy in chronic neurodegenerative diseases is due to the ability of memantine to block low tonic levels of pathological activation of NMDA receptors and secondary excitotoxicity with mild membrane depolarization, while allowing physiological activation following synaptic release of glutamate. Additionally, NMDA receptor hypofunction, or under-excitation, has been proposed as a contributing factor in the etiology of senescent memory changes in normal aging and in various psychiatric disorders. Experimental NMDA hypofunction is associated with abnormal memory, cognitive and behavioral function. Hypofunctional NMDA receptors can down regulate neural mechanisms that regulate encoding and consolidation of memory and produce clinical syndromes that include the core features of psychosis, as well as dissociation. Sustained and severe NMDA hypofucntion is associated with a neurotoxic process with classical neuropathological features. Thus, memantine may upregulate the function of NMDA receptors in various conditions and provide improved neuronal function as well as providing neuroprotection.

Finally, NMDA receptor function has been implicated in the modulation of blood brain barrier (BBB) function. Excessive NMDA stimulation can produce increased BBB permeability that would allow potential neurotoxins to gain access to the CNS tissue, producing additional neuronal dysfunction and demyelination to disturbances from excessive NMDA receptor stimulation. Severe NMDA activity may increase NO activity and peroxynitrite formation that would further alter BBB permeability. These mechanisms may have implications in the pathophysiology of neurological diseases such as meningitis or sepsis.

Pharmacology

Memantine is completely absorbed from the gastrointestinal tract and TTP (time-to-peak concentration) occur with in 6-8 hours after oral intake. The plasma clearance half-life (t_{1/2}) is usually between 60-100 hours and steady-state plasma levels occur in approximately 21 days. The protein binding is between 42-45%. Excretion is renal and consists of unchanged memantine as well as its hydroxylated metabolites. Urine pH has been found to influence the renal excretion of memantine with an alkaline urine producing reduced renal excretion and renal clearance. Memantine easily penetrates the blood brain barrier but the CSF (cerebrospinal fluid) concentration is decreased by 20-50% due to albumin binding. In humans, doses of 20 mg per day of memantine produce serum levels which range from 0.5-1.0 μM. Dosing is commonly initiated at 10 mg per day (5 mg BID or 10 mg QD) and usually titrated to a dose of 10 mg po BID, however doses of 30 mg per day or higher may be tolerated in certain clinical conditions. In patients treated with 10-30 mg Memantine per day, plasma levels of 0.4-1.0 μM have been measured. Brain microdialysis with in vivo recovery indicate that free rat brain concentrations are 20-30% lower in plasma, whereas CSF sampling in human subjects showed 30-40% lower concentrations. The adverse events of Memantine are dose-related and include nausea, dizziness, and restlessness. In patients with a predisposition to seizures, memantine may decrease the threshold for seizures, especially at higher doses. Drug interactions that may accentuate adverse reactions include barbiturates, neuroleptics, L-dopa, dopamine agonists, and amantadine. There was no adverse drug interaction when memantine was combined with AchE (acetylcholinesterase inhibitors). Memantine is contraindicated in delirium, severe renal insufficiency, and currently should be used with caution in pregnancy. No induction of HSP (heat shock protein) or neuronal vacuolization and necrosis have been observed in animals studies.

At clinically therapeutic doses, memantine reaches a brain ECF concentration in the range of its affinity for the NMDA receptor. At levels of 1-10 μM, the mechanism of action of memantine is specific for antagonism of the NMDA receptor and does not affect other ligand-gated or voltage-gated channels. Importantly, these concentrations (6-10 μM) do not attenuate LTP in hippocampal slices or alter the function of the postsynaptic excitatory currents. The therapeutic concentration that produces efficacy in Parkinson's disease is less than 2 μM. At concentrations greater than 100 μM, memantine interacts with multiple receptors including the D2, AMPA, kainate, alpha 1, alpha 2, and 5-HT re-uptake. Memantine preferentially blocks the NR2C and NR2D subunits, has intermediate potency at NR1A/2B and weak effects at NR2A of the NMDA receptor, which may explain its efficacy in certain neurological conditions.

Pathophysiology

Mechanism of neurodegeneration via stimulation of the NMDA receptor include as least: acute high glutamate concentrations, chronic exposure to subacute elevations of glutamate, decreased neuronal energy in the presence of elevated or normal levels of glutamate, and additional mechanisms of agonist stimulation such as inflammation, cytokines or quinolinic acid (QUIN). An NMDA hypofunction theory has also been proposed in which aging and disease processes produce neurological symptoms and neurodegeneration by conditions that under-stimulate this receptor. With advanced aging, the number of NMDA receptors, subunit composition and binding kinetics are decreased or altered which may contribute to the severity and course of a particular disease.

Normal glucose metabolism, ATP production, and ATPases function which are critical in generating a resting membrane potential that maintains the voltage-dependent Mg++ block of the NMDA receptor channel are decreased in mitochondrial dysfunction. In neurological diseases with decreased neuronal energy or mitochondrial dysfunction, a reduction in the resting membrane potential relieves the Mg++ block and renders the neurons susceptible to physiological concentrations of glutamate. The lack of the Mg++ block enables persistent excitatory stimulation, opening of the channel, and initiation of an intracellular calcium cascade which produces neuronal damage. Thus, under certain conditions, glutamate is converted from a neurotransmitter to a neurotoxin. This mechanism is blocked by memantine which restores the physiological activation of NMDA receptors and is believed to produce the symptomological cognitive enhancement observed in clinical trials of dementia, at doses up to 20 mg ad day for durations of 4 to 6 weeks. Thus, the simultaneous blockage of the neurotoxic effects of NMDA activation at concentrations with no effect on normal physiological function, contributes to the unique efficacy and safety of memantine.

In hippocampal slices, removal of Mg++ impairs neuronal plasticity or LTP, while the addition of memantine normalized synaptic functioning, at relevant human brain concentrations (1-μM) by substituting for the absent Mg++ ions. In hippocampal slices, NMDA depressed synaptic transmission in CA1 and also caused a moderate reduction in LTP induction/expression which was antagonized by memantine. Thus, under conditions of tonic activation of NMDA receptors, memantine reversed deficits and learning and synaptic plasticity (LTP). Memantine prevents hippocampal damage, convulsions and cell death induced by the ICV (intracerebral injection) of QUIN, a potent NMDA agonist and neurotoxin. Additional neurophysiological mechanisms and evidence of neuroprotection in vitro include: (1) an increase of the CA1 pyramidal cell spike by 100%; (2) reversal of deficits in LTP induction following reduction of Mg++ with the restoration of LTP; (3) prevention of neuronal ganglion cell death in primary culture when administered 4 hours after NMDA neurotoxicity; and (4) prevention of apoptosis induced by gp120 from the HIV-1 virus in cortical cell cultures.

In animal studies, the chronic ICV infusion of an endogenous NMDA agonist QUIN, produced memory deficits which were blocked by simultaneous infusion of memantine. Memantine prevented the decrease in cortical choline uptake sites with QUIN and has shown efficacy in providing neuroprotection in inflammatory models of neurological disease. With NMDA injection into the rat NBM (nucleus basalis magnocellularis), choline acetyltransferase levels in cortical target areas were decreased. In addition, lesions of the NBM produced by mitochondrial toxins (3-NP or 3-nitroproprionic acid) are inhibited by memantine which also significantly attenuated striatal lesions by malonate, a model for mitochondrial neurological diseases, suggesting potential clinical efficacy. With lesions of the entorhinal cortex, memantine reversed the learning impairment within 3 days and normalized learning within 8 days. Thus, memantine has revealed neuroprotective activity and produced positive effects on learning/LTP at clinical therapeutic relevant doses and concentrations. In summary, memantine has been shown to: (1) prevented learning deficits in various animal models of ischemic and neurodegenerative diseases; (2) prevented the loss of basal forebrain cholinergic neurons; (3) produced cognitive enhancement in rats with NMDA lesions of the nucleus basalis magnocellularis; (4) provided neuroprotection against injections of P-amyloid into the CA1 hippocampal region; (5) increased the duration of the LTP in older animals; (6) prolong the duration of LTP in vivo and improved memory retention in the Morris maze test; and (7) significantly reduce infarct size up to 2 hours after induction of hypoxia/ischemia in immature and adult rats. Conversely, it has been reported that NMDA antagonists increase neuronal damage in mature brain neurons undergoing slowly progressive degeneration while providing neuroprotection to in models of rapidly progressing neuronal death. Thus, progressive neurodegeneration in the basal ganglia induced by the mitochondrial toxin (3-NP) or in the hippocampus by traumatic brain injury (TBI) was enhanced by NMDA antagonists, including memantine. Parallel treatment with memantine and 3-NP produced more neurological impairment and increased mortality with both a reduction in volume (11.5%) and enhanced neuronal density drop-out (26%) in the striatum, leading these authors to caution against long-term monotherapy of NMDA antagonists in humans with TBI or progressive neurological diseases. The mechanism has been attributed to a caspase-mediated induction of programmed cell death. However, since low-intensity stimulation of the NMDA receptor increases intracellular calcium and protects cells from caspase-mediated death, the allowance of baseline NMDA stimulation by memantine should have prevented this form of cell death. In addition, a speculative hypothesis is that baseline or physiological glutamate simulation of the NMDA receptor may produce a trophic function in the mature brain neuron.

With normal brain aging, the NMDA receptor system becomes progressively hypofunctional which may contribute to normal age-related decreases in memory and learning performances. In addition, various psychiatric diseases have been proposed to have NMDA hypofunction as a contributing mechanism. Decreased memory performance is common in drug usage and severe hypofunction of the NMDA receptor (i.e., PCP) can produce symptoms such as hallucinations, delusions, poverty of speech and though, agitation, emotional withdrawal, decreased motivation and memory, and dissociation. Acute, sub-anesthetic doses of ketamine produced delayed memory recall and decreases in verbal and nonverbal memory in normal subjects. In addition, ketamine can cause “emergence reactions” in patients awakening from anesthesia as well as a mild, dose-dependent clinical syndrome that includes cognitive and dissociative effects. Thus, NMDA hypofucntion affects neural mechanisms that regulate encoding, processing, and consolidation into long term memory. It has been further postulated that NMDA hypofunction may cause disruption of neuronal cytoskeleton structures; alter GABA, glutamate and acetylcholine homeostasis; and reduce both recurrent feedback inhibition and feedforward function of neural circuits involved in memory.

In conclusion, memantine gains rapid access to the open channel at the NMDA receptor at the initiation of pathological over activity and thereby attenuates its progression. Its high index of therapeutic efficacy and safety is due to the ability to block tonic low level pathological activation of NMDA receptors by agonists and mild membrane depolarization in chronic neurodegeneration diseases while simultaneously allowing physiological NMDA activation following synaptic release of glutamate. Neuroprotection can be defined as any treatment strategy of treating a neurological disease by attenuating acute or chronic neuronal injury or cell death, preventing progressive neurological degeneration, and preventing apoptosis, and will here-in refer to blocking of the open-channel at the NMDA receptor.

Recent 18F-memantine PET scan studies in normal volunteers revealed a homogenous distribution in human brain. The authors concluded that while the receptor-rich regions such as the striatum and frontal cortex could be well imaged, the homogenous distribution of the ligand in the brain made it unsuitable for the PET imaging of the NMDA receptor. We disagree with the statement that white matter lacks NMDA receptors, since these have been reported. In addition, since 18F-memantine has a homogenous binding pattern, it application to specific diseases (Huntington's disease which shows a 50% reduction in NMDA receptor density, cerebellar disease, mild cognitive impairment, post-ischemic syndromes) would show decrements in the areas of the brain most affected. Thus, we predict 18F-memantine would be able to diagnose both asymptomatic and pre-clinical disease states as well as certain neurological conditions that have distinct pathology. Other NMDA antagonists such as Felbamate could be radioactively labeled to diagnose certain neurological diseases by the binding pattern.

### OBJECTS OF THE INVENTION

One of the objectives of the present invention is to provide compositions and methods for the prevention and/or decrease in progression of acute or chronic neurological disorders that involve excessive activation of the NMDA receptor, which compositions are relatively non-toxic, have high degree of effectiveness and continue to produce a therapeutic response over a prolonged period of time.

Another object of the invention is to provide compositions and methods for the treatment of acute and chronic neurological disorders in humans that involve excessive activation, increased or decreased NMDA receptor density, abnormal NMDA subunit composition, abnormal NMDA receptor binding kinetics, or hypofunction of the NMDA receptor.

Yet another object of the present invention is to provide compositions and methods effective to control or attenuate acute or chronic neurological disorders utilizing compounds that act as non-competitive antagonists of the open channel of the NMDA receptor, either at the Mg++ site or at an independent site.

Still another object of the present invention is to provide compositions and methods effective to prophylactically treat or prevent progression of acute or chronic neurological disorders.

A further objective of the invention is to provide a method for the attenuation of neuronal death in diseases or neurological diseases (presymptomatic) that cause loss of cognitive function, by preventing neuronal death by excessive activation or hypofunction of the NMDA receptor.

A further objective of the invention is to provide a method for the attenuation of apoptosis or necrosis in diseases or neurological diseases (presymptomatic, acute, subacute or chronic) that cause loss of neuronal death, by preventing neuronal death by excessive activation or hypofunction of the NMDA receptor.

An additional objective of the invention is to provide a method for the treatment of diseases or neurological diseases (presymptomatic, acute, subacute or chronic) by normalizing NMDA receptor function in conjunction with other standard medical treatments for that particular disease.

Finally, an objective of the invention is to provide a method for the treatment of diseases or neurological diseases (presymptomatic, acute, subacute or chronic) that combine preventing hypofunction (under stimulation) or excessive NMDA receptor activation at the open-channel with other forms of neuroprotection: glycine-site NMDA inhibitors, inhibitors of glutamate release or synthesis, AMPA and kainate inhibitors, polyamine inhibitors, inhibitors of NO (nitric oxide) synthesis, GABA inhibitors, anti-oxidants, acetylcholinesterases, nootropic drugs, calpain inhibitors, or the addition of various nerve growth factors.

Moreover, it is a further object of the present invention to provide methods for the attenuation and treatment of diseases or acute and chronic neurological disorders by providing intravenous, transdermal, rectal, oral routes (including sustained or extended release formations) or modified drug delivery systems (such as lipid emulsion or crystal technology) of administration that prevent excessive activation or hypofunction of the NMDA receptor by acting at the open-channel.

### SUMMARY OF THE INVENTION

The present invention is directed to pharmaceutical compositions which are open-channel antagonists of the NMDA (N-methyl-D-aspartate) receptor complex, specifically memantine (a 1-amino-3,5-dimethyl-adamantane hydrochloride). Memantine is hypothesized to bind at the Mg++ site or multiple other sites in the channel of the NMDA receptor. The invention relates to methods of use for the acute, chronic and prophylactic treatment of neurologic and neurodegenerative diseases, attenuation of acute or chronic neuronal damage in neurological disease (“neuroprotection”), and prophylaxis of neurological diseases. Neurological diseases may involve excessive stimulation of the NMDA receptor, hypofunction of the NMDA receptor, up- or down regulation of the NMDA receptor, and abnormal subunit structure or function of the NMDA receptor. The invention relates to oral, controlled or sustained release, intravenous, rectal, transcutaneous or other preparations such as lipid emulsion or crystal technology. The trade name is Akatinol Memantine® (Merz and Co.) The invention also relates to the compounds felbamate, acamprosate, and MRZ 2/579.

Therapeutic uses of the Compound of the Invention

Memantine (1-amino-3,5-dimethyl-adamantane hydrochloride), and other open-channel antagonists of the NMDA receptor are useful in the treatment of multiple neurological diseases in which there is NMDA receptor hypofunction, abnormal NMDA receptor density, abnormal NMDA receptor subunit composition, or excessive stimulation of the NMDA receptor by at least glutamate, quinolinic acid, glycine, and NMDA agonists. In addition, memantine will be administered to prevent acute and delayed apoptosis and necrosis. Another compound is Acamprosate which has multiple mechanisms of action (NMDA receptor antagonist, voltage-dependent Ca++ channel blocker and alteration of immediate early gene and glutamate receptor expression), MRZ 2/579 (a moderate affinity uncompetitive NMDA antagonist) and Felbamate (a glycine-site NMDA antagonist with properties of open-channel antagonism, AMPA antagonism, GABA enhancement, and Na+ channel blocker).

Neuroprotection in Epilepsy

Epilepsy may be defined as a neurological disease characterized as a paroxysmal, self-sustaining and self-limited cerebral dysrhythmia, genetic or acquired in origin, and either physiologic or organic in mechanism. Epilepsy is classified by clinical and EEG criteria into generalized seizures, partial or focal seizures, plus various other specific epileptic syndromes. Current drugs utilized in the treatment of epilepsy function as prophylactics against the clinical symptoms of epilepsy (the reduction and control of epileptic seizures) rather than as neuroprotection against the neurological sequela of seizures and epilepsy such as brain atrophy, mesial temporal sclerosis, psychiatric and cognitive dysfunctions. Up to 20-30% of seizures are intractable despite maximum medical therapy while brain atrophy and degeneration occur even when current drugs are able to control the clinical manifestations of epilepsy or seizures.

Epilepsy may cause brain damage by glutaminergic NMDA mechanisms. Elevated levels of glutamate have been measured by microdialysis in human brains who suffered from intractable complex partial seizures. These increased levels of glutamate were observed at resting levels during inter-ictal periods (between seizures) and prior to the development of a seizure. We hypothesize that both chronic and acute intermittent elevations of glutamate during seizures, post-seizures (ictal) and during inter-ictal periods produce excessive NMDA receptor stimulation in epileptic patients. This results in at least neuronal degeneration, gliosis, brain and hippocampal atrophy observed in epileptic patients. In addition, the NMDA receptor may involved in the etiology of various seizures and the phenomena of kindling. Finally, epilepsy and seizures may produce abnormal quantities or function of NMDA receptors which may further exacerbate seizures and promote neurodegeneration. Recent evidence that brain gliomas secrete glutamate and that seizures resulting from brain tumors eventually become intractable, provide additional evidence for a glutaminergic etiology of intractable seizures. In addition, excessive glutamate secretion by the brain tumor may produce the secondary brain atrophy, by induction of apoptotic mechanisms, often observed in these patients.

Memantine has been reported to have minimal efficacy as an anti-convulsant or in the treatment of epilepsy. However, while other standard anti-epileptic drugs may control and suppress the clinical manifestations of seizures, their mechanism of action may not produce neuroprotection from chronic basal increases or post-ictal elevation of glutamate, as well as other NMDA agonists. According to the subject invention, the addition of memantine to all patients with seizures, intractable seizures, or complex partial seizures (even when other drugs have efficacy in seizure control) will function in neuroprotection or the prevention of neuronal degeneration and brain atrophy. The NMDA antagonist is also to be administered to patients with seizure disorders to prevent delayed cellular necrosis in patients that may have controlled seizures, uncontrolled seizures, intractable seizures or status epilepticus. Memantine will also function to produce cognitive enhancement in patients with chronic intractable seizure. An unexpected finding was an increased in cognitive enhancement in patients with intractable seizures, who underwent baseline and follow-up neuropsychological examinations, when a glycine-site NMDA antagonist was added to a standard treatment.

Memantine, administered chronically in oral doses of 5-100 mg/day, advantageously 10-30 mg/day (serum levels ranging from 0.25-2.0 μg/ml) is efficacious providing neuroprotection and in attenuating neuronal gliosis, necrosis and atrophy in epilepsy. Memantine will be administered (1) concomitantly with other standard anti-convulsants (including glycine site antagonists) that function to suppress the various forms of clinical seizures; with (2) concomitant oral magnesium supplements that also acts to suppress NMDA over-activity and increases the efficacy of memantine; and (3) with both standard anti-convulsants and oral magnesium to increase the efficacy of memantine; (4) added to the standard treatment to improve cognitive dysfunction in patients with chronic and intractable epilepsy and (5) used with other future therapies such as calcium channel blockers, partial β and γ secretase inhibitors, anti-oxidants, anti-inflammatory drugs, caspase inhibitors, neurotrophins, or neural stem cell implantation.

Familial Alzheimer's Disease (FAD)

Alzheimer's disease (DAT) is a chronic progressive neurological disease producing clinical dementia and cortical atrophy with up to 15% of cases being familial or genetic. Those skilled in the art will recognized that FAD is a distinct neurological disease from sporadic Alzheimer's disease. Neurofibrillary tangles (NFT) and neuritic plaques (SP) comprise the major neuropathological lesions. Significant genetic risk factors include: those encoding APP (chromosome 21) and presenilin-1 or -2 mutations in familial autosomal dominant disease; ApoE which may function as a time-dependent susceptibility gene depending on the quantity; and possibly α-2 macroglobulin, a deletion mutant. A prominent theory of the etiology of DAT is excessive, abnormal amyloid deposition (Aβ42) in the brain. Aβ is formed from APP (β-amyloid precursor protein) by secretase cleavage. With presenilin mutations, elevations of Aβ42 and Aβ40 are found brain, plasma and skin fibroblasts while PS-2 mutations are implicated in enhanced neuronal apoptosis. Gene deletion of PS1 in mice produced an embryonic-lethal phenotype which included neurodevelopmental abnormalities of the forebrain. PS1 has been reported to regulate the neural threshold to excitotoxicity (over expression of PS1 variants increased the vulnerability of neuronal damage while a reduction resulted in neuroprotection). Thus, Aβ42 accumulation and diffuse plaques produces local microglial activation, cytokine release, reactive astrogliosis and inflammation (complement cascade activation) which produces altered calcium homeostasis and selective neuronal death. Excessive amyloid deposition may produce induction of glutamate toxicity via the NMDA receptor. Thus neuronal death and atrophy occurs in areas of the brain that have a high density of NMDA receptors, such as the hippocampus and cerebral cortex. Additional evidence that the NMDA receptor plays a significant role in cognitive dysfunction is acute kainate toxicity, wherein 25% of the patients who accidently consumed domoic acid, had memory loss, some profound and permanent. Finally, enhanced apoptosis in DAT has been postulated due to dysregulation of apoptotic genes or apoptotic cellular mechanisms. Those skilled in the art will recognize that the hypothesis of amyloid-induced glutamate neurotoxicity as the prime etiology of DAT or FAD is not a current widely accepted theory.

The NMDA receptor is composed of an NR1 subunit, which is obligatory for channel function, and NR2 subunits (A to D). Memantine acts on these subunits with varying potency but preferentially acts on the NR2C and NR2D subunits compared to the NR2A/NR2B subunits. The NR2B subunit is concentrated in the cortex and hippocampus and regulates channel gating, Mg++ dependency, and functions in LTP (long-term potentiation), a form of synaptic plasticity, which is required for the formation of autobiographical memory and spatial learning. NR2B expression is downregulated during normal aging (and possibly in neurodegenerative diseases) and correlates with the gradual shortening of the EPSP (excitatory post-synaptic potential) duration of the NMDA channel. Increasing the expression of NR2B subunits in the forebrains of transgenic mice improved both memory and learning. This was correlated with an increase in the size and duration of the EPSP and enhancement of LTP. Thus, the NR2B is critical in gating the age-dependent threshold for plasticity and memory function. We propose that the modest action of memantine on the NR2B subunit may partially explain its reported clinical efficacy in various dementia syndromes.

Memantine has been shown to have efficacy in decreasing the rate of cognitive decline in patients with moderate and severe Alzheimer's disease and improves their functional capacity and activities of daily living. We propose that memantine will have efficacy when administered to pre-clinical patients at risk for FAD (by genetic analysis, abnormal metabolism by PET scanning, ApoE levels, or CSF tau levels) as well as mild, moderate, and severe cases of FAD. Prior memantine patents claim (Lipton U.S. Pat. No. 5,334,618 and U.S. Pat. No. 5,614,560) in DAT which refers to the sporadic form, but no pathophysiological rationale is offered and no method of treatment is claimed. Those skilled in the art will recognize that FAD is a distinct neurological disease from DAT. A prior patent on memantine (Olney U.S. Pat. No. 5,958,919) for the treatment of DAT uses a theory that NMDA antagonists can cause hypofunction of NMDA receptors that triggers neurotoxic side effects and that co-administration of “safener” drugs are required to prevent toxic side effects. Thus, this theory holds that memantine and other NMDA antagonists has the possibility of making the disease worse and therefore a contingency for withdrawal of the drug is proposed. We disagree with the contention memantine would produced additional hypofucntion of NMDA receptors but suggest they would in fact normalize the receptor to a more functional state and by inducing genes, possibly normalize the receptor density in disease states. In a case where a glycine-site antagonist (with specificity for the NR2B subunit) was administered to a patient with dementia from vascular factors including hypertension, leukoariosis, lacunar infarcts, and a right parietal hemorrhage; an unexpected finding was an increase in almost all areas of the neuropsychological exam at 6 months that was correlated with the ability of improved ADL (activity of daily living). Additionally, those skilled in the art will recognize that all of our claims are outside of the scope of pre-mild, moderate, or severe Alzheimer's disease.

Memantine, administered chronically in oral doses of 5-100 mg/day, advantageously 10-30 mg/day (serum levels ranging from 0.25-2.0 μg/ml) is efficacious in attenuating the progression of dementia in patients with mild, moderate and severe DAT and FAD. Memantine will also have efficacy in (1) treating DAT when used in combination with a glycine-site antagonist, (2) treating FAD when used in combination with a glycine-site antagonist, (3) treating both DAT and FAD when used in combination with an acetylcholinesterase inhibitor and in (4) FAD patients at risk (increased ApoE4, elevated CSF tau levels) for developing dementia but clinically normal, as monotherapy or with various combinations of glycine-site NMDA antagonists or acetylcholinesterase inhibitors. In addition, memantine may also be used with other future therapies such as calcium channel blockers, partial β and γ secretase inhibitors, anti-oxidants, anti-inflammatory drugs, caspase inhibitors, neurotrophins, or neural stem cell implantation.

Mild Cognitive Impairment (MCI)

Mild cognitive impairment refers to the transitional zone or time period between normal aging and mild dementia. However, those skilled in the art will recognize that there is no convincing evidence that this is a specific disease or DAT. Criteria for the diagnosis of MCI may include subjective and objective memory impairment, normal cognitive and activities of daily living (ADL), and the absence of any specific criteria for dementia. The cognitive impairment may be amnestic (memory) or involve any other isolated cognitive domain that is greater than expected for normal aging. The patient and family may have insight into the impairment, but the patient is still able to function adequately with ADL. The objective memory function detected by neuro-psychological tests usually 1.5 SD below the average performance of individuals with similar age and education. MRI of the brain may reveals mild atrophy of the hippocampus and entorhinal cortex while neuropathologic studies can reveal some early features of DAT. However, neocortical SP and entorhinal NFT were observed in subjects with no detectable cognitive decline. Thus, while subjects with MCI have a condition that differs from normal aging and are likely to progress to dementia at an accelerated rate, not all patients progress to dementia. Finally, most subjects with MCI that convert to dementia or DAT have elevated levels of CSF tau protein.

We hypothesize that MCI represents the earliest detectable cognitive brain dysfunction due to glutaminergic toxicity producing chronic over-stimulation of NMDA receptors. This pathological process produces progressive neuronal cell death and apoptosis. In addition, patients with mesial temporal atrophy with MCI may have a more advanced form of the disease. We classify MCI into subtypes: (1) a pure clinical syndrome, including the amnestic variant, diagnosed solely on neuropyschological criteria, and (2) a pure radiological form with mesial temporal sclerosis or atrophy on brain MRI without clinical evidence, (3) a clinical syndrome combined with hippocampal atrophy and (4) a clinical syndrome, with or without radiological evidence, in patients with risk factors such as ApoE4 and elevated CSF tau.

A prior patent (Olney U.S. Pat. No. ______) for pre-Alzheimer's disease does not overlap with the diagnosis of MCI. Those skilled in the art will recognize that MCI is not considered as DAT since only a portion will eventually develop DAT while pre-Alzheimer's disease is a mild form of DAT. In addition, his theory of hypofucntion of NMDA receptors as an etiology and postulation of the potential deterioration of the disease is not congruent with our theory or our observed clinical results.

Memantine, administered chronically in oral doses of 5-100 mg/day, advantageously 10-30 mg/day (serum levels ranging from 0.25-2.0 μg/ml) is efficacious in attenuating early neuronal gliosis, necrosis and atrophy in subtypes of MCI and delaying or preventing the clinical conversion of the subtypes of MCI subtypes into dementia or DAT. Memantine may be utilized in combination with acetyl-esterase inhibitors or glycine-site NMDA antagonists. As well, memantine may be combined with future therapies such as calcium channel blockers, partial β and γ secretase inhibitors, anti-oxidants, anti-inflammatory drugs, caspase inhibitors, neurotrophins, or neural stem cell implantation.

Non-Alzheimer Dementias

Cognitive decline may occur in various other neurological diseases which have dementia as a symptom and which may have either a genetic predisposition (chromosome 17), contain Lewy bodies or tau proteins. For example, mutations of tau occur in families with FTDP-17 (frontal temporal dementia linked with Parkinson's disease). This syndrome is characterized by widespread NFT formation associated with tau, in the absence of amyloid deposits. Thus, abnormalities of tau structure and function produces progressive, severe neuronal degeneration and death. Additional dementing illnesses include frontotemporal dementia, progressive supranuclear palsy, Pick's disease, corticobasal degeneration, alcoholic dementia, (DLB) dementia with Lewy bodies, Picks' disease, thalamic dementia, hippocampal sclerosis, Hallervorden-Spatz, multiple system atrophy, tauopathies, subacute aterioscleroitic encephalopathy (Binswanger's disease), amyloid angiopathy, vasculitis, prion diseases, and paraneoplastic syndromes. Those skilled in the art will recognize that these diseases are not Alzheimer's disease or MCI condition. We propose that a contributing factor to the dementia of these diseases involve a glutamate excitatory process that produces excessive NMDA stimulation, resulting in neuronal cell death. The stimulation of the NR2B receptor, although not of major potency, by memantine will enhance cognitive function and decrease the rate of cognitive decline.

Memantine, administered chronically in oral doses of 5-100 mg/day, advantageously 10-30 mg/day (serum levels ranging from 0.25-2.0 μg/ml) is efficacious in attenuating early neuronal gliosis, necrosis and atrophy in these neurological diseases and delaying or preventing the progressive cognitive dysfunction to dementia in these syndromes. Memantine may be combination with acetyl-esterase inhibitors and glycine-site NMDA antagonists. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Downs Syndrome (Trisomy 21)

Down's syndrome (DS) is a chromosomal abnormality that occurs with a frequency of 1 in 700 births. Mild to severe retardation is universal and the disease has many pathological features, such as senile plaques and neurofibrillary tangles, with Alzheimer's disease. A lifelong over expression of APP occurs in DS which results in overproduction of both Aβ40 and Aβ42 peptides. Thus, diffuse plaques of Aβ42 occur as early as 12 years of age and progressively accumulate with most patients developing full Alzheimer's pathology after the 40^{th} year of life. The temporal progression of these lesions occur between 20-50 years. DS exemplifies the importance of Aβ42 deposition as a seminal event in the development of DAT pathology since the appearance of NFT is delayed until 25-40 years of age. The accrual of these brain lesions is associated with additional loss of cognitive and behavior function at 35 years of age.

The role of such NMDA agonists such as glutamate and quinolinic acid in DS are unclear. Excessive amyloid deposition may produce abnormal Ca++ homeostasis by glutamate toxicity via the NMDA receptor and therefore memantine, with neuroprotective properties, may be a useful treatment for early DS to prevent the neuronal degeneration by progressive accumulation of Aβ and NFT. Memantine would attenuate any NMDA-mediated injury, decrease NMDA induced apoptosis, and attenuate progressive cognitive dysfunction in DS.

Memantine would be administered most advantageously orally after the diagnosis of DS as a neuroprotectant agent against potential excessive NMDA stimulation. Memantine, administered chronically in oral doses of 5-100 mg/day, advantageously 10-30 mg/day (serum levels ranging from 0.25-2.0 μg/ml) is efficacious in controlling the progressive neurological symptoms and sequela of DS. Memantine administered acutely and chronically, as monotherapy or in conjunction with current standard medical treatments, will be efficacious for the treatment of the acute and chronic neurological complications of DS. Memantine may be used in combination with acetyl-esterase inhibitors and glycine-site NMDA antagonists. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Cognitive Enhancement in Normal Senescence

Normal NMDA receptor function is required for learning and memory and with advanced normal aging, the NMDA receptor transmitter system (NR-1 and NR2B) becomes hypofunctional with decreases in the number of NMDA binding sites (cortex>hippocampus) as well as variable age-related changes in glycine-site binding. NMDA receptor hypofunction is postulated to produce excessive release of glutamate and acetylcholine in the cerebral cortex. These age-related decrements in the number of NMDA receptors and the gradual shortening of the EPSP may contribute to mild decrements of learning and memory in normal aging. The NR2B subunit of the NMDA receptor is critical in the in gating the age-dependent plasticity threshold for plasticity and memory function. This subunit is downregulated during normal aging while the EPSP (excitatory post-synaptic potential) duration, an index of the function of the subunit, shortens with normal aging. Thus, by lengthening the EPSP of the NR2B subunit during normal aging, we propose that memantine will maintain learning and memory in normal healthy aging humans and those with diseases that may interfere with cognition. Evidence supporting our hypothesis is that memantine has been shown to prolong the duration of the LTP in the hippocampus in older animals. In addition, we also theorize that memantine may possibly increase or attenuate the normal decrease in the density of NMDA receptors that occurs in the aging process as well as reverse the deleterious effects of NMDA hypofunction. Finally, in subjects with no evidence of cognitive dysfunction, pathological evidence of neocortical SP and entorhinal NFT has been documented, suggesting a neurochemical and neuropathological process that exists prior to the development of MCI (mild cognitive impairment). A prior patent coving the topic of pre-DAT is not synonymous with our hypothesis. This hypotheses attributes the etiology of DAT to hypofunction of NMDA receptors and posits that the addition of an NMDA receptor to such patients will cause more NMDA receptor blockade that will result in worsening of the clinical syndrome and the production of more neuronal damage. Our hypothesis is that NMDA receptors are down-regulated by glutamate and cytokines and that the addition of an NMDA antagonist will normalize the function of the NMDA receptor by allowing its physiological functioning while preventing any pathological functioning. Our theory is supported by the unexpected findings of documented cognitive enhancement in chronic complex partial seizures and dementia from multiple vascular factors.

Memantine, administered chronically in oral doses of 5-100 mg/day, advantageously 10-30 mg/day (serum levels ranging from 0.25-2.0 μg/ml) is efficacious in maintaining and improving memory and learning in normal senescence. Memantine may attenuate the normal decrease in the EPSP of the NMDA receptor during aging and prevents deficits in learning and memory and prevent the development of MCI syndromes. Memantine may also be used in combination with acetyl-esterase inhibitors and glycine-site NMDA antagonists. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Meningitis

Despite maximum bactericidal efficacy of antibiotic treatment in bacterial meningitis, the morbidity and mortality still remain consistently high. Half of the patients who survive meningitis suffer long-term neurological sequela, specifically with learning and memory deficits, in addition to motor deficits, seizures, and hearing loss. Morphological evidence of necrotic and apoptotic neuronal cell death suggests a preferential susceptibility of the hippocampus and dentate gyrus.

Viral replication in cerebral endothelial cells can increase BBB permeability by both direct and immune mediated damage. The secondary elevation of cytokines (such as TNF-α, IL-6) can also directly increase BBB permeability and cause both demyelination and neuronal damage by an increase in NO synthesis, histamine and peroxynitrite production. Viral, bacterial and chronic meningitis infections all increase CSF levels of quinolinic acid, aspartate, glutamate levels and other inflammatory mediators. Thus, elevated NMDA agonists, cytokines and inflammation may significantly contribute to the neurological mortality and morbidity observed in meningitis by excessive NMDA receptor stimulation. Toxicity is also due to the release of bacterial products and up-regulated host inflammatory mediators, such as interleukin 1β, which is a potent pro-inflammatory cytokine. The cytotoxicity of bacterial free CSF suggests that inflammatory mediators such as glutamate and TNF-α are apoptotic in meningitis. In meningitis, the degree of apoptosis was increased by glucocorticoids plus antibiotic but decreased with monoclonal antibody plus antibiotic. The inhibition of glutamate uptake in the hippocampal astrocytes by glucocorticoids treatment may be an important role in the neuronal injury of the dentate gyrus during meningitis. Finally, oxidative injury contributes to intracranial complications and brain damage by ROS and peroxynitrite that produces cytotoxic effects, including the initiation of lipid peroxidation and induction of DNA breakage.

In humans, neuronal apoptosis in the dentate gyrus (density 1-19/mm²) has been observed in bacterial meningitis. The density of apoptotic neurons was dependent on the interval between the onset of symptoms of meningitis and death but was not related to neuronal damage in other parts of the brain or prior treatment with steroids. Apoptotic cell death (3-11%) occurred in the granular cell layer of the dentate gyrus, but not CA1 region of the hippocampus, within 24 hours suggesting that apoptotic cell death occurs in the initial phase of bacterial meningitis. In patients with meningitis, elevated CSF cell count and increases in concentrations of glutamate correlated with clinical severity as well as both morbidity and mortality. In experimental pneumoccal meningitis, a broad-spectrum caspase inhibitor has been shown to provide neuroprotection by preventing hippocampal neuronal cell death and leukocyte influx into the CSF. Hippocampal neuronal death from apoptosis was directly due to the inflammatory response in the CSF. Consistent with this observation is the neuroprotective effect of both kynurenic acid (a caspase inhibitor) as well as an anti-inflammatory treatment in animal models of bacterial meningitis.

In summary, meningitis causes nervous tissue damage by multiple mechanisms including direct bacterial toxicity, host inflammatory reaction, increased oxidative stress, free radicals, excitatory amino acids, and caspases. Since learning defects are frequently observed in survivors of bacterial meningitis, strategies to reduce the degree of apoptotic neurons in the dentate gyrus will decrease the frequency of neurologic sequela in surviving patients. We thus hypothesize that an NMDA antagonist, such as memantine, added to the acute and chronic conventional treatment of meningitis would result in a decrease in the neurological morbidity and mortality.

Memantine, administered intravenously in acute and chronic meningitis, followed by chronic oral doses of 5-100 mg/day, advantageously 10-30 mg/day (serum levels ranging from 0.25-2.0 μg/ml) to patients with acute and chronic meningitis is efficacious in preventing the neurological morbidity and mortality in meningitis. Memantine will be administered, preferably intravenously, prior to any steroid treatment to prevent and decrease the adverse neurological sequela caused by the elevations of glutamate. The length of treatment will be determined by efficacy parameters such as clinical course, CSF values and neuroimaging studies. Memantine will also be administered concomitantly with standard medical therapy (antibiotics) and other treatments which decrease the toxicity of glutamate and inflammatory mediators (glycine-site NMDA inhibitors, AMPA antagonists, NOS inhibitors, caspase inhibitors etc).

Sepsis and Septic Encephalopathy (SE)

We define septic encephalopathy as a dysfunction of mental state, level of consciousness, and cognition that is initiated by an infectious or septic process extrinsic to the brain. Sepsis is clinically diagnosed with evidence of infection, fever, abnormal vital signs, and decreased end-organ perfusion. SE can be classified as: (1) initial, the clinical neurological status prior to multiple organ failure and (2) late, a more severe neurological dysfunction that is associated with multiple organ failure, hypotension, and decreased perfusion of organs. The various syndromes of SE (sepsis syndrome, septic shock, and ARDS) are the most common cause of mortality in the ICU. We postulate that the neurological dysfunction and coma in systemic blood infections are also contributory to morbidity and mortality (as are cardiac, pulmonary and renal dysfunction). With the clinical diagnosis of sepsis, EEG is sensitive in confirming SE while the severity of EEG abnormalities predicts both morbidity and mortality. In addition, EEG abnormalities are observed in the presence of a normal clinical evaluation.

The pathogenesis of SE is caused by septic inflammation causing pro-inflammatory mediators to be released by leukocytes which then damage both endothelial cells and astrocytes. The inflammatory mediators act directly on neural tissue or by a secondary cytotoxic response by brain cells to these mediators. The release of inflammatory mediators (TNF-α and interferon γ) produce abnormal endothelial permeability, decreased cerebral blood flow (CBF), reduced cerebral oxygen uptake, and increased intracranial pressure (ICP). Abnormal cerebral endothelial permeability results in dysfunction of the BBB structure and function as well as neuronal mitochondrial dysfunction. Astrocyte dysfunction interferes with local regulation of CBF, substrate transport, energy levels, and metabolism. SE also produces dysfunction in neuronal systems (i.e., the RAS) causing cognitive dysfunction. Finally, abnormal neurotransmitter metabolism (increased serotonin turnover in the raphe nuclei and decreased NA in the locus ceruleus), abnormal brain levels of amino acids and the production of “false transmitters” also contribute to the clinical expression of SE. While enhanced GABA function has been postulated to produce impaired motor function and decreased cognitive levels, the role of glutamate or other NMDA agonists has not been evaluated in SE. Our hypothesis is that sepsis causes an increased CNS inflammatory reaction and glutamate levels which contributes to the clinical syndrome of SE by altering neuronal function and increasing the permeability of the BBB.

Cardiac tissue has a specialized neural conduction system for rapid conduction and regulation of cardiac rhythmicity. Cardiac tissue contains NMDA, AMPA and kainate receptors (specific for Glu R2/3, Glu R 5/6/7, KA 2 and NMDAR1) localized to cardiac nerve terminals, ganglia, conducting fibers, and at atrium myocardiocytes. The stimulation of cultured rat myocardial cells by L-glutamate produces an increase in the intracellular Ca++ oscillation frequency. These effects are not thought to be metabolic in nature but may be important in cardiac function and cardiotoxicity. Thus, glutamate may play a significant role in both cardiac physiology and pathology.

NMDA receptors are located in the alveolar walls, bronchiolar epithelium and endothelial lining. NMDA receptor activation in perfused, ventilated rat lungs triggered acute injury that was marked by increased pressures required to ventilate and perfuse the lungs as well as by high-permeability edema. These pulmonary injuries were prevented by MK-801, reduced by Mg++ and were nitric oxide (NO) dependent. Thus, excessive pulmonary NMDA receptors located in the lung may contribute to acute lung edema in ARDS, a frequent complication of systemic sepsis. Finally, renal NMDA R1 receptors are preferentially located in the cortical structures such as glomeruli, convoluted and distal tubules and hence function in electrolyte and water homeostasis. We hypothesize a role for glutamate in renal dysfunction in SE.

Additional evidence that cardiorespiratory and ANS dysfunctions are associated with excitotoxins are the frequent occurrence of palpitations and arrhythmias that occur after ingestion of MSG (mon-sodium glutamate) as well as the observation that acute human domoic acid toxicity (a kainate agonist) produces profuse respiratory secretions, unstable blood pressure, and cardiac arrhythmias. Thus, the etiology of cardiovascular and systemic abnormalities in sepsis may have a partial but significant neurogenic etiology mediated by the NMDA receptors. Consistent with this hypothesis is that after bilateral injection of KA and NMDA into the paraventricular nucleus, KA elicited pressor responses, tachycardia and sudden cardiac death while NMDA produced cardiovascular stimulation. Neither of these changes were prevented by a peripheral β-blocker. Importantly, after 48 hours, KA but not NMDA, produced myocardial pathology including intramyocardial hemorrhages, hyaline myocardial necrosis and predominantly mononuclear necrosis. These observations suggest that abnormal stimulation of the NMDA receptors in the hypothalamus can produce abnormal systemic effects, similar to those observed in SE, by acting via the sympathetic nervous system. We postulate that sepsis also increases serum glutamate levels as well as other inflammatory mediators that contribute to systemic cardiac, vascular and other peripheral tissue pathology.

Memantine may have efficacy in multiple organ systems in systemic sepsis by attenuating neuronal dysfunction, cardiac toxicity, renal dysfunction and pulmonary dysfunction (ARDS, edema) by acting of the NMDA receptors located in these tissues. Decreased NMDA mediated Ca++ neurotoxicity as well as the prevention of downstream mechanisms (activation of NO, calpain etc.) will attenuate symptoms and complications of sepsis. We hypothesize that elevated levels of both CSF and serum glutamate, cytokines and quinolinic acid cause excess stimulation of both central and peripheral NMDA receptors, which result in cell dysfunction and death. In animal models, memantine has been shown to reverse the neurologic effects of quinolinic acid and also prevent neurologic dysfunction by inflammatory mechanisms. Thus, memantine may attenuate coma in septic patients and prevent morbidity and mortality by its antagonistic action of the NMDA receptor.

Memantine, administered intravenously acutely and then chronically in oral doses (including via nasogastric tube) of 5-100 mg/day, advantageously 10-30 mg/day (serum levels ranging from 0.25-2.0 μg/ml) is efficacious in attenuating central neuronal dysfunction as well as secondary systemic complications, including morbidity and mortality. Memantine will be administered concomitantly with standard medical therapies including glycine-site NMDA antagonists and AMPA receptor antagonists. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

CNS Vasculitis

CNS vasculitis is an inflammatory disorder of the cerebral arteries that occurs in various genetic and autoimmune diseases (Sjogren's disease, rheumatoid arthritis). Neurological sequela include stroke, seizures, and dementia. Elevations of CSF quinolinic acid have been found to correlate with the degree of brain damage on MRI, dementia and the clinical severity in Sjogren's disease. Cerebral autosomal dominant arteriopathy with subcortical infarcts and leukencephalopathy (CADASIL) is a genetic disorder (chromosome 19) with recurrent mid-life ischemic episodes that result in neurological impairment and cognitive dysfunction.

An immune-mediated abnormality appears to be related to an immunological attack of a subtype (GluR) of a glutamate receptor in rare form of epilepsy. Antibodies to GluR3 in animals developed seizures and inflammatory lesions of the cortex, similar to the syndrome of Rasmussen's encephalitis, a rare progressive syndrome of intractable seizures. Thus, glutamate receptor abnormalities may contribute to the pathogenesis of epilepsy syndromes and inflammatory brain degeneration.

Memantine, administered chronically in oral doses of 5-100 mg/day, advantageously 10-30 mg/day (serum levels ranging from 0.25-2.0 μg/ml) is efficacious in attenuating the effects of quinolinic acid in autoimmune CNS vasculitis. In addition, memantine will decrease the rate of cognitive decline as well as the progression of chronic ischemic brain lesions in vasculitides. Memantine will be administered concomitantly with other standard medications such as steroids, immune suppressants, and glycine-site NMDA antagonists. In addition, memantine may also be combined with future therapies such as calcium channel blockers, partial β and γ secretase inhibitors, anti-oxidants, anti-inflammatory drugs, caspase inhibitors, neurotrophins, or neural stem cell implantation.

Schizophrenia

Schizophrenia (SCZ) is a chronic, heterogenous, psychotic disorder with clinical symptoms including withdrawal from reality, delusions, hallucinations, inappropriate affect, and abnormal behavior. SCZ is a neurodevelopmental disorder with disturbances of brain structure and function evolving over time as evidenced by brain imaging. Autopsy studies reveal abnormal brain morphology in specific regional brain regions. Clinical psychiatric manifestations are currently theorized to be mainly due to a metabolic dopamine disorder. Genetic influences are implicated in the expression of schizophrenia since the brain atrophy on MRI studies in patients with schizophrenia are observed in their monozygotic twins.

Glutamate and NMDA dysfunction has been hypothesized in the pathophysiology of schizophrenia. Phencyclidine (PCP) intoxication, which acts as a non-competitive antagonist at a site in the channel of the NMDA receptor, mimics the clinical expression of schizophrenia. In addition, both ketamine and MK-801 also produce the symptoms of schizophrenia and will exacerbate symptoms in schizophrenic patients. A proposed mechanism of action for ketamine is an increased glutamate release acting on the prefrontal cortex AMPA receptors, which then induces a hyperdopaminergic state. Based on knockdown mice (where the expression of NMDA receptors are decreased) a hypothesis of reduced NMDA activity or “hypofucntion” in schizophrenia has been proposed. A selective interaction between glutamate and dopaminergic mechanisms involving the NMDA receptors in the limbic forebrain has also been proposed. Thus, abnormal glutamate transmission and NMDA receptor dysfunction may produce an increase in dopamine metabolism, resulting in the clinical expression of schizophrenia. Finally, glycine-site NMDA antagonists have been reported to be moderately effective in alleviating the negative symptoms and enhancing cognitive dysfunction while glycine has been reported to show efficacy. We posit that the effect of glycine in schizophrenia may be to modulate the NMDA receptor to a more normal functional state.

Memantine, administered chronically in oral doses of 5-100 mg/day, advantageously 10-30 mg/day (serum levels ranging from 0.25-2.0 μg/ml) is efficacious in the treatment of both the clinical symptoms of schizophrenia as well as any glutamate induced neuronal degeneration and brain atrophy. Memantine may be administered with standard medical therapy for the treatment of schizophrenia (DA receptor blockers) or additional glutamate antagonists such as glycine-site antagonists, AMPA antagonists, or glutamate release inhibitors. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Drug and Opiate Addiction

A major characteristic of addiction is the proclivity for recidivism after a period of abstinence. Dopamine (DA) transmission in the nucleus accumbens (NA) is involved in the reward process. DA receptor agonists are self-administering and modulate cocaine-seeking behavior while D₁ DA antagonists in the NA reduces the re-inforcing efficacy of cocaine. Glutamate transmission in the NA is associated with a behavioral sensitization while AMPA receptor inhibition prevents both the expression of sensitization and increased glutamate transmission following acute cocaine administration in sensitized rats. Behavioral sensitization to psychomotor stimulants correlates with abnormalities in the mesoaccumbens dopamine (DA) system. These include at least DA autoreceptor subsensitivity in the ventral tegmental area and D₁ receptor supersensitivity in the nucleus accumbens (NA). Finally, other illicit drugs such as ecstacy (MDMA) have a predilection for destroying serotonin brain neurons.

In animal studies, both NMDA antagonists (non-competitive and competitive) and AMPA antagonists prevented both cocaine sensitization and receptor alterations. Glutamate transmission from the medial prefrontal cortex to the mesoaccumbens DA system was critical for the induction of cocaine sensitization and receptor correlations. Glycine binding site NMDA antagonists and inhibitors of nitric oxide synthetase (NOS) have been reported to attenuate the development of morphine tolerance and even reverse established tolerance or dependence. The modulation of tolerance and dependence by glutamate antagonists without effecting the analgesic effect of morphine suggests prevention of neuronal plasticity associated with the adaptive changes mediated by the NMDA/NO cascade. Within neurons expressing both the NMDA and mu opiod receptor, the magnitude of NMDA receptor-mediated inward current is enhanced by mu opiod agonists. Mu receptor activation may function by removing the Mg++ block, allowing increased NMDA activation and the subsequent formation of NO. This cascade alters gene expression and produces neuronal plasticity, resulting in both tolerance and dependence. The latter neurochemical events decrease the analgesia cascade effect of morphine. Thus NMDA antagonists can interfere with the phenomena of drug tolerance without having a direct effect on the analgesic effect of mu opiod stimulation. The stimulation of glutamate receptors in the NA was shown to augment the reinforcing effect of cocaine, supporting the concept that increased glutamate transmission in the NA is involved in facilitating the relapse to cocaine seeking behavior.

The symptoms of drug tolerance, dependency, addiction and withdrawal that occur in both opiod addicts and chronic pain patients may be partially mediated by the NMDA receptor complex. In animal studies, a glycine-site receptor antagonist revealed efficacy in decreasing withdrawal symptoms and eliminating opiate drug addiction. In contrast, the results of memantine in eliminating symptoms of drug withdrawal and addiction in animal studies have been conflicting and usually negative, which may reflect an inadequate treatment period. When a patient chronically addicted to heroin and cocaine was administered a glycine-site antagonist, an unexpected finding was a gradual decrease in addiction, tolerance and dependence that resulted in a drug free state for several years with no evidence of recidivism even after the drug was discontinued.

Memantine, administered chronically in oral doses of 5-100 mg/day, advantageously 10-30 mg/day (serum levels ranging from 0.25-2.0 μg/ml) is efficacious in the treatment of acute and chronic opiod tolerance. The concomitant use of memantine and analgesics in acute and chronic pain will decrease the potential of opiod tolerance and physical dependence. The administration of memantine to patients with chronic tolerance and dependence, in conjunction with current standard medical therapy (i.e., Naloxone or Acompatase) is also proposed in the treatment of illicit drug addiction. Memantine IV in patients with acute opiod or illicit overdose is also proposed. In addition memantine may also be combined with future therapies such as calcium channel blockers, partial P and y secretase inhibitors, anti-oxidants, anti-inflammatory drugs, caspase inhibitors, neurotrophins, or neural stem cell implantation.

Alcoholic Diseases

Alcohol (ETOH) addiction is a complex pathological behavior governed by ETOH-conditioned cues and involving long-lasting adaptations in brain-reinforcement systems. The spectrum of alcoholic diseases includes acute ethanol intoxication, withdrawal symptoms, withdrawal seizures, delirium tremens, blackouts, Wernicke's (WE) syndrome, and alcoholic dementia. Ethanol appears to inhibits the release of multiple transmitters including serotonin, dopamine, norepiniephrine, glutamate, aspartate, and GABA as a consequence of its interaction at the NMDA receptor. Alcohol affects glutaminergic transmission by at least interfering with fast excitatory transmission, potentiating excitotoxicity, and impairing neurodevelopment. The mechanism(s) are controversial but appear to involve interaction at the NMDA glycine-site. Thus, glycine has been shown to (1) reverse the inhibitory effect of ethanol on NMDA-stimulated dopamine release, (2) decrease ethanol-mediated inhibition of NMDA-stimulated calcium influx, and (3) reduce glycine enhancement of Glu production of cGMP in the cerebellar granule cells. However, in the hippocampus, neither the inhibition of the NMDA-activated current of ethanol nor the NMDA-stimulated norepinephrine release was glycine dependent. Specifically, ethanol does not increase binding at the glycine-site which suggests a modulatory mechanism at the NMDA channel ionophore.

Acute ETOH administration causes significant decreases of both aspartate and glutamate levels in the midbrain and brainstem and also decreases glutamate concentration in the hippocampus. Ethanol also increases the density of glutamate receptors in the cerebral cortex, striatum, thalamus, and hippocampus within 10 to 24 hours. Increased binding sites for NMDA receptors (NMDA R1) in the hippocampus may be a compensatory mechanism for overcoming ethanol-mediated inhibition. In single-channel recordings, ethanol decreased the probability of NMDA channel opening as well as the mean open time of the channel. The observation of increased intracellular Ca++ concentration is consistent with an increased number on NMDA receptors. In contrast, during the chronic alcohol state, glutamate release is decreased while glutamate uptake and tissue concentration are increased. During the ETOH withdrawal state, glutamate synaptic release, uptake, and tissue concentration are increased, although there are significant regional variations in the degree of increased glutamate metabolism. Importantly, the number of NMDA receptors is increased during acute ETOH withdrawal, which we hypothesize contributes to the clinical expression of this disorder.

The electrical current generated by NMDA activation is reduced by ETOH in a concentration dependent manner suggesting that intoxicating concentrations of ETOH correlate with the inhibition of NMDA currents. Acute ethanol ingestion also inhibits dopamine and norepinephrine release and has been shown to inhibit the excitation of the locus ceruleus noradrenergic neurons by both glutamate and NMDA. Following the acute withdrawal from chronic ETOH administration, the locus ceruleus (LC) has increased sensitivity to both NMDA and quisquilate and displays functional hyperactivity from an up-regulation of glutamate receptors in the locus ceruleus. Thus, ethanol has indirect effects on this catecholaminergic system that is modulated by the NMDA receptor. We hypothesize that this interaction may account for the autonomic instability and behavioral agitation observed in both alcohol withdrawal and delirium tremens. Thus, the effects of acute and chronic ETOH differ. While acute ETOH ingestion protects against glutamate induced degeneration and NMDA-induced convulsions by decreasing free intracellular calcium, chronic ingestion increases NMDA receptor density in the LC resulting in both elevated excitatory neurotransmission and noradrenergic activity with ethanol withdrawal.

ETOH toxicity produces the amnestic disorder (Wernicke-Korsakoff syndrome) due to the malabsorption of thiamine. Pathological features include necrotic lesions of the mamillary bodies, brainstem and thalamic regions. In animal models, extracellular glutamate is significantly increased in the ventral posterior thalamus, while intracellular glutamate and aspartate concentrations decrease. Consistent with a glutamate dysregulation hypothesis, NMDA antagonists have been shown to prevent lesions in the medial thalamus and mamillary bodies as well as protecting against working memory deficits in animals. In this disorder, Impaired cognition and blackouts can be explained by chronic ETOH inhibition of NMDA transmission resulting in decreased LTP, which impairs hippocampal function, since ETOH attenuates LTP in the hippocampus by its inhibitory effect on NMDA receptors.

Three distinct alcohol syndromes of uncomplicated ETOH withdrawal, ethanol withdrawal seizures, and delirium tremens may be due to the up-regulation of NMDA receptors and catecholamine activation. Acute ETOH withdrawal produces neuronal hyperexcitability by alterations in GABA, voltage-gated Ca++ channels, and glutamate/NMDA activity. ETOH withdrawal induces decreased mesolimbic dopamine activity, increased glutamate in the nucleus accumbens (NA), and increases in nuclear c-fos expression. An increase in NMDA receptor density produces an amplification of the catecholamine effects as well as permanent memory deficits in WE, since an up-regulation of NMDA receptors increases neuronal vulnerability to excitotoxicity. Thus, receptor rich and glutamate dependent NMDA regions (such as the hippocampus, cerebral cortex, and cerebellum) are preferentially vulnerable to ETOH toxicity and produce the neural basis for global cognitive impairment in alcoholic dementia. Animal analysis of NMDA receptors after chronic ETOH administration has reported increased binding sites and alteration in function and subunit composition. In contrast, a post-mortem study NMDA ligand binding in human alcoholic brains has revealed no change in the amount of receptors. Distinct NMDA receptor subunit compositions, in particular the NR1B/NR2B are reported to be more sensitive to ETOH than NMDA R1 and R2C channels, with non-NMDA receptors having the highest ethanol sensitivity. Conversely, in the fetal alcohol syndrome, chronic ETOH has been postulated to decrease NMDA receptor density and metabotropic (mGLu) receptor function producing deleterious effects on neurodevelopment.

Decreased alcohol consumption in mice lacking β-endorphin suggest an important ETOH-opiod interaction, possibly by influencing early gene expression. In patients at risk for alcoholism, ETOH consumption dose-dependently increased plasma levels of β-endorphin suggesting that a blockade of endogenous opiod systems can influence ETOH intake. Naltrexone, which blocks opiod-receptors and inhibits ETOH-induced DA release in the nucleus accumbens (NA), is a controversial FDA approved adjunctive medication in the treatment of alcohol disorders. Thus, activation of the endogenous opiod system may play a crucial role in ETOH reinforcement, long-term neuronal plasticity, and in craving.

In summary, the indirect effects of ETOH on the catecholamine system via the NMDA receptor may account for the ANS instability, behavioral agitation, and psychosis seen in ETOH withdrawal and delirium tremens. We postulate that NMDA antagonists would produce efficacy by decreasing the alcohol-reinforcement and deprivation effect, suppressing c-fos expression in the hippocampus and NA, and modulating post-synaptic activation of glutamate transmission. Memantine may have efficacy in ETOH disorders by preventing neuronal plasticity by NMDA mechanisms that would decrease neuronal excitability in acute withdrawal and prevent permanent neuronal damage in chronic ETOH conditions. The subunit specificity of memantine for NR2C and NR2D predict an excellent efficacy profile since ETOH shows preference for these subunits. A recent report that memantine reversed cognitive effects in a patient with alcoholic dementia but did not totally reverse metabolic deficits (by PET scanning) is consistent with this hypothesis. Memantine, when administered in conjunction with NMDA receptor antagonists specific for the NR2B receptor or naltrexone, may have additional efficacy in treating of ETOH disorders. No double blind clinical studies showing the efficacy of memantine have been published.

Memantine, administered chronically in oral doses of 5-100 mg/day, advantageously 10-30 mg/day (serum levels ranging from 0.25-2.0 μg/ml) is efficacious in the treatment of acute and chronic alcohol dependence and tolerance, alcohol withdrawal syndromes, delerium tremens, Wenicke' syndrome, and alcohol dementia. The administration of memantine to patients with chronic tolerance and dependence, in conjunction with current standard medical therapy, is also proposed. Memantine may also be combined with Naltrexone, acompotase, glycine-site NMDA receptors, NR2B specific NMDA antagonists and AMPA receptor antagonists. Memantine IV in patients with acute alcohol withdrawal symptoms, alcohol withdrawal seizures, and delerium tremens is also proposed. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Multiple Sclerosis (MS) and Demyelinating Diseases

MS is classically defined as a primary demyelinating disease with secondary cortical dysfunction of unknown etiology but caused by an interaction between genetic (MHC and HLA) and environmental factors. Environmental factors clearly affect the expression of the disease, since specific genes are neither essential nor sufficient for disease susceptibility. Although the risk is increased in MZ twins, concordance for white matter disease is only 30%, suggesting that multiple genes interact to increase susceptibility. The disease may be relapsing-remitting pattern (85%) or primary progressive MS with less common syndromes including acute MS, acute disseminated encephalomyelitis, neuromyelitis optica, transverse myelitis, Balo's concentric sclerosis, etc. The lesion is presumed to be both primarily inflammatory and demyelinating, although axonal loss is an important factor that correlates with progressive and irreversible disability. Pathological findings predominate in the optic nerve, periventricular white matter, brain stem and spinal cord.

Since viral infections are implicated in up to 25% of episodes of acute clinical relapses, various viral hypotheses including concepts such as latent viruses activation, molecular mimicry, or antigenic similarity between microbes and tissues have been proposed. Current theory postulates T cells activation by exposure to virus, penetration of the BBB, and in genetically predisposed persons, misidentification of normal myelin antigens as ‘virus’ with subsequent injury. Contemporary doctrine postulates a thymic abnormality that activates T cells followed by the immunological activation of T-lymphocytes, macrophages and microglia that produces white matter inflammation.

Pathologically, multi-focal sites of myelin destruction, perivascular-lymphocytic cuffing and a variable degree of oligodendroglial loss are seen in acute cases, with gliosis, axonal transection and neuronal and axonal loss less prominent. T-cells may damage oligodendrocytes by inducing proinflammatory cytokines (IFN-γ and TNF-α). The importance of axonal pathology in the evolution of pathology and as a determinant in disability has recently been recognized. The observation that chronically demyclinated axons have an increased density of sodium channels indicates that repair mechanism(s) other than remyelination by oligodendrocytes is important in restoring axonal conduction.

Neuroimaging techniques have contributed important advances to the pathology and progression of MS. MRI evidence of axonal loss include hypointense lesions on T1-weighted images as well as abnormalities on normal appearing white matter (NAWM). Reduced N-acetyl aspartate (NAA) levels in lesions, a marker of neuronal or axonal loss, have been reported using MR spectroscopy (MRS). In progressive MS, clinical progression of the disease occurs in the absence of new demyelinating lesions or prolongation of central neural conduction time. Thus, axonal degeneration may occur independent of and prior to new formation of demyelinating lesions, as well as with chronic demyelinating lesions. The latter finding can be interpreted as a neuronal degeneration in MS being the primary lesion which causes a secondary demyelination.

Using MTI (magnetization transfer imaging) techniques in MS patients, low ratios (MTR) have been shown to reflect myelin damage and reveal diffuse tissue damage in both NAWM and NAGM (gray matter) in MS. Significantly, MTR reductions were detected in NAWM prior to lesion formation, decreased in NAWM in MS in the absence of T2-visible lesions, and most severe in areas adjacent to focal T2-weighted MS lesions. In PPMS (primary progressive) a subtle but more widespread damage in NAWM is a major contributor to neurological impairment. Thus, in NABT (normal appearing brain tissue), abnormalities in the MTR was the only factor significantly associated with cognitive impairment in MS while cognitive impairment was proportional to the degree of NAGM damage.

In diffusion MRI, the ADC (apparent diffusion coefficient) reflects a diffuse loss of cellular structural barriers to water molecular motion and can quantify the amount of tissue damage of an MS lesion. Widespread subtle changes in the ADC were detected in NAWM. The ADC of MS lesions were increased compared to NAWM, diffusion values of NAWM in MS were lower than controls, T2 visible lesions were lower than NAWM, while hypointense T1 lesions had the lowest values. Quantitative MRI diffusion values in MS correlated with clinical variables (disability, disease duration) and cerebral atrophy while histograms were able to differentiate between secondary progressive and relapse-remitting MS patients. Thus, the peak height of the ADC histogram was a more specific marker for both axonal loss and clinical fixed disability when compared to measures of cerebral atrophy.

Using T2-weighted MR images (T2WI), cortical and subcortical gray matter (GM) hypointensities in MS brains were related to disease duration, clinical course, and degree of neurological disability. T2 hypointensities correlated with total brain atrophy, total T2 (white matter) lesion load, 3^{rd} ventricular enlargement, and parietal lesions. The GM hypointensity on T2WI was postulated to reflect pathologic iron metabolism and deposition in MS. Third ventricle enlargement in MS patients correlated with disability, depression, cognitive disability, decreased QOL, brain hypometabolism, parenchymal lesions, and global atrophy suggesting a major role for the subcortical structures in the clinical expression of MS. These results support our theory that fatigue and cognitive decline may be independent of demyelinating lesion and more dependent on axonal lesions. They also argue for an early initiation of treatment with NMDA antagonists to both prevent and treat fatigue and cognitive decline.

Additional support is provided by MR spectroscopy that revealed axonal damage in both lesions and surrounding NAWM. Longitudinal monitoring of NAA suggests axonal damage is an early event, decreases of NAA in NAWM are transient in the acute phase of demyclination, and axonal loss contributes to disability. Importantly, alterations in phosphorylation and a substantial loss of axon density occurred peripheral to demyelinating lesions, confirming a more significant wide-spread involvement of NAWM in MS pathology. Thus, axonal loss is a major contributor to disease progression in PPMS, a variant with no clinical relapse-remission episodes, few MRI-T2 lesions, minimal Gd++ lesion enhancement, and minimal accruement of additional white matter lesions during disease progression. Evidence of axonal loss in NAWM by MRS supporting the hypothesis that axonal loss may occur prior to demyclination PPMS. Thus we hypothesize that the neurological deficits of PPMS are primarily due to axonal loss due to glutamate receptor dysfunction while RRMS produces neurological deficit as a result of both axonal loss and demyelination (as well as incomplete recovery of relapses from incomplete remyelination).

EAE (experimental autoimmune encephalopathy) is an animal model of neurological inflammatory disorders which has some relation to MS and glutamate dysfunction has been postulated to contribute to the pathogenesis. In the spinal cord of EAE, both myelin and neurons that were subjected to lymphocytic attack had less damage and degeneration when AMPA receptors were blocked. The number of neuronal vacuoles containing lymphocytes that were observed to undergo apoptosis in the spinal cord correlated with the clinical stages of the disease. T lymphocytes were shown to enter the neurons and initiate inflammation during EAE, with the degree of spinal cord lymphocytic infiltration correlating with the time course of the disease. Treatment with AMPA antagonists at the onset of neurological decline also resulted in a profound reduction in neurological deficits in EAE in the absence on the effects on neuroinflammation (perivascular cuffs). In EAE, memantine (Wallstrom, 1996) failed to have any effect on decreasing CNS inflammation, interferon gamma (IFN-γ), lymphocytic proliferation, or systemic immunity. Quinolinic acid, an NMDA agonist, is elevated in the spinal cords of EAE animals while increased CSF concentrations of glutamate have been reported in MS patients. Thus, both glutamate and AMPA antagonists are effective in suppressing inflammatory damage within the white matter, decrease the axonal damage, ameliorate symptoms and prevent clinical relapses when treatment is initiated at the onset of paralysis in EAE. These antagonists do not influence immune response to myelin antigens but appear to protect oligodendrocytes from immune-mediated damage and thus decrease axonal damage. We hypothesize that the inflammatory response (either primary or secondary) in MS and EAE increases glutamate release in both brain microglia and macrophages, activating glutamate receptors and producing neuronal destruction.

In humans, active MS lesions reveal high-level glutaminase expression of both macrophages and microglia in close proximity to dystrophic axons. Glutamate elevation from both activated leukocytes and microglial cells is combined with a reduction of glutamate transport and metabolizing enzymes (GDH, GDS) beyond the lesion. Elevated glutaminase expression correlates with markers of axonal damage (NF-H) while decreased glutamate transporters (GLT-1) expression occurs in oligodendrocytes surrounding active MS lesions. Finally, GS (glutamine synthetase) and GDH (glutamate dehydrogenase) activity were absent from both active and chronic silent MS lesions suggesting permanent metabolic alterations. Since ionotropic receptors are located on myelinated axons, they are susceptible to glutamate toxicity. Axonal damage produces an increase in myelin lesional activity, suggesting a mechanism where the degree of demyelination can be secondary to the degree of glutamate induced axonal degeneration. We propose that abnormal glutamate homeostasis contributes to both axonal and oligodendroglial pathology in MS due to increased glutamate production, alterations in glutamate transporters, and decreased glutamate metabolizing enzymes. In addition, axonal damage by glutamate may be the primary lesion or etiology in MS.

Glutamate receptors and transporters are expressed in macroglial cells and indicate that oligodendrocytes may also participate in glutamate uptake. Glutamate transporter subtypes are located in neurons, glia, cerebellum and retina which are the most common areas of damage in multiple sclerosis. In optic nerves, acute kainate application produced inflammation similar to MS plaques while chronic exposure produced atrophy of optic nerves. Thus, KA toxicity may produce either apoptosis or necrosis of oligodendroglia depending on the intensity and duration of the exposure. A brief infusion of excitotoxins induces apoptotic oligodendroglial death while prolonged infusion produces oligodendroglial death, demyelinating plaques, and axonal damage as well as inflammation, necrosis and atrophy. Taken together, these results suggest that glutamate dysfunction plays a pivotal role in lesion formation in MS.

Oligodendrocytes are especially vulnerable to glutamate receptor activation because while they contain high permeability glutamate receptors to Ca++, they do not express several intracellular Ca++ binding proteins present in neurons. They also modulate extracelluar glutamate levels by transporters, and thus acute and chronic elevations of glutamate may contribute to the development of demyelinating lesions. In addition, the activation of glutamate receptors in microglia increases the release of the pro-inflammatory TNF-α which are toxic to oligodendrocytes. In humans, CSF QUIN concentrations are increased while the amount of glutamate has been associated with the severity and course of the disease.

Activated microglia upregulate and release neurotoxic inflammatory mediators resulting in excessive glutamate receptor activation, producing both oligodendroglial and axonal damage and death. The presence of glutamate receptors on both myelin sheaths and myelinated neurons provide a mechanism for direct glutamate toxicity. Thus, axonal damage may occur by direct excitatory receptor mechanisms axonal damage, secondary damage from demyclination due to excitotoxicity mechanisms, and autoimmune mechanisms. The degree of damage in any individual may be modified by the degree of apoptotic genes, since in animals with less BcL (an anti-apoptotic gene) there is less neuronal damage in MS. Taken together, these results suggest that a primary neuronal process may occur prior to myelin injury. We hypothesize a neurogenic etiology of MS where individuals have a genetic predisposition to a primary neuronal gray matter glutamate abnormality that produces increased glutamate metabolism and inflammatory processes. Upregulation of cytokines and glutamate, which result in over activate glutamate receptors on both neurons and oligodendrocytes, produce increased intracellular calcium and cell death in both white and gray matter. Glutamate dysfunction can also alter the permeability of the blood brain (especially with fever and infection) barrier that allow a secondary brain inflammatory infiltration and result in myelin pallor and demyclination.

Current therapy of MS is only partially effective despite the use of multiple anti-inflammatory, immunosuppressive and immune modulatory treatments. β-interferons have a modest benefit in delaying clinical progression, although the duration of the benefit remains unclear, and mechanism(s) unknown although immune suppression is postulated. The possible role of MHC II (major histocompatibility complex) in genetic susceptibility to MS may explain the relative efficacy of Copaxone (co-polymer I) whose mechanism of action is blocking MHC presentation of brain specific myelin fragments. Longitudinal MRI studies also support the modest benefit of β-interferons which delay clinical and MRI evidence of progression in secondary progressive MS.

In conclusion, cortical brain damage occurs frequently and cognitive dysfunction, depression and fatigue are common symptoms in MS. NMDA receptors are located in both the cortex and oligodendrocytes and an inflammatory process occurs with upregulation of cytokines, increased quinolinic acid and glutamate. Brain atrophy including both cortical and white matter is common in multiple sclerosis which we posit is due to chronic overstimulation of glutamate receptors. We hypothesize that patients with MS may have genetic abnormalities in the quantity, structure or function of NMDA receptors which contribute to their susceptibility and clinical expression of MS. Thus, an NMDA antagonist would have efficacy in protecting both the cortex and white matter from the lesions of MS. By decreasing the deleterious effects of increased glutamate and quinolinic acid, memantine would have a neuroprotective effect by protecting both the neuronal and myelinated structures in the cortical and subcortical areas that contain the NMDA receptor. Memantine in combination with AMPA antagonists (i.e., topiramate) or glycine-site NMDA antagonists may have efficacy in preventing neuronal and oligodendroglial degeneration and thus prevent and ameliorate symptoms of MS such as fatigue and cognitive dysfunction.

Memantine, administered chronically in oral doses of 5-100 mg/day, advantageously 10-30 mg/day (serum levels ranging from 0.25-2.0 μg/ml) is efficacious in the treatment of all acute, chronic or progressive forms of CNS or spinal multiple sclerosis. Memantine will be administered at the initial diagnosis of MS (intravenously with an acute attack) and maintained chronically for the duration of the disease to prevent cognitive decline, brain atrophy and demyelination. Memantine will be administered prior to the acute or chronic administration of steroids to prevent the deleterious effects of increased glutamate levels by the mechanism of decreased glutamate uptake. Memantine will be administered acutely and chronically in conjunction with current standard medical immune treatments (i.e., steroids, Avonex, Copaxone, B-interferons etc.) for multiple sclerosis. Memantine will be also administered acutely and chronically in concomitantly with medications known to block the AMPA/kainate receptor, the glycine-site NMDA receptor, or both receptors. In addition memantine may also be combined with future therapies such as calcium channel blockers, partial P and y secretase inhibitors, anti-oxidants, anti-inflammatory drugs, caspase inhibitors, neurotrophins, or neural stem cell implantation.

The Leukodystrophies (LD) and Adrenoleukodystrophy (X-ADL)

Leukodystrophies can be defined as a predominant progressive disease of central myelin in which a genetic metabolic defect produces confluent destruction, maldevelopment of the central white matter, inflammatory reactions, and secondary gray matter dysfunction resulting in cognitive dysfunction. Of the dozen known LD diseases, twelve can be diagnosed precisely using non-invasive techniques, while the molecular defect has been isolated in nine diseases. ADL is an X-linked recessive disorder of myelin metabolism resulting in seizures and progressive dementia in males. Diverse clinical phenotypes reflect two distinct pathological mechanisms: an inflammatory demyelinating process that produces a rapid progressive fatal cerebral X-ADL and a slowly progressive, distal axonopathy that produces adrenomyeloneuropathy in young adults. In all forms of X-ADL, very long-chain fatty acids (VLCFA's) accumulate in tissues and body fluids due to an impairment in peroxisomal lipid metabolism. MRI findings of T1-contrast enhancement produces a highly predictive prognosis with the MRI abnormalities usually preceding symptoms in X-ADL patients with cerebral involvement. In addition, brain MRI has prognostic value in relation to the age of the patient and has efficacy in selecting patients for bone marrow transplantation, an effective therapy in some patients. Pathological evidence of inflammation suggest that glutamate and quinolinic acid may contribute to the pathology of ADL. Apoptosis of oligodendrocytes is an additional mechanism of neuropathology that occurs in human cerebral X-ADL.

Alexander's disease has several forms: infantile (megalencephaly, seizures, developmental retardation, death) and juvenile (ataxia, spasticity, bulbar signs) in which the white matter abnormality is predominantly frontotemporal. Intracellular inclusions in astrocytes (Rosenthal fibers) contain GFAP and stress proteins. Canavan's disease is an infantile syndrome of white matter spongy degeneration with macrocephaly, retardation, and seizures. The enzyme aspartoacylase is deficient and causes an accumulation of NAA (N-acetyl aspartate) in the brain and body fluids. Other diseases include cerebrotendinous xanthomatosis, Krabbes, and metachromatic LD.

An NMDA antagonist may have efficacy as a neuroprotectant in LD and X-ADL by decreasing the neurotoxic effects of inflammatory mediators and NAA. Memantine may provide neuronal protection, decrease the rate of cognitive dysfunction, and possibly myelin degeneration by antagonizing the NMDA receptors on myelinated fibers.

Memantine, administered chronically in oral doses of 5-100 mg/day, advantageously 10-30 mg/day (serum levels ranging from 0.25-2.0 μg/ml) is efficacious in attenuating the deleterious effects of inflammatory mediators of both cortical and myelin NMDA receptors, including the process of demyelination and cognitive dysfunction in all leukodystrophies.

Memantine may also be used in combination with glycine-site NMDA antagonists and AMPA antagonists. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Fatigue

Fatigue is a subjective sensation of weakness or state of increased subjective discomfort, decreased efficiency with minimal exertion, and characterized clinically by reduced physical endurance. Fatigue is a common complaint of patients with chronic diseases (depression, Parkinson's disease, multiple sclerosis etc) as well as fibromyalgia and the enigmatic chronic fatigue syndrome (CFS). CFS is defined as prolonged fatigue with multiple somatic symptoms and marked disability in the absence of organic illness or psychiatric disease. The primary complaint of persisting or relapsing fatigue may be accompanied by decreased cognition, insomnia, headache, paresthesias and ataxia. It has been postulated that altered BBB permeability, neural cell dysfunction and altered neuronal transmission contributes to the pathophysiology of CFS. CFS may have an autoimmune or viral component to its etiology producing cytokine upregulation and subsequent glutamate dysfunction and NMDA over activity, which contributes to the fatigue syndrome. However, a recent study failed to reveal any upregulation of gene expression of enzymes in antiviral pathways in patients with CFS.

MRI brain studies in CSF revealed frontal white matter abnormalities occurred more frequently than in controls, possibly reflecting edema, gliosis or demyelination. These brain abnormalities contribute to the neurological symptoms of cognitive impairment, vestibular dysfunction, and ataxia. Abnormal cerebral and brain stem perfusion on SPECT scans further indicate neuronal dysfunction. The etiology of CFS-FM-PTSD has been postulated to be due to excessive stimulation of NMDA receptors by physical trauma or psychological stress which subsequently elevates NO and increases the levels of the oxidant, peroxynitrite. The latter factors induce BBB breakdown that is further increased by the upregulation of inflammatory cytokines resulting in neuronal dysfunction.

An NMDA antagonist will provide efficacy by providing symptomatic relief of fatigue as well as neuroprotection from potential glutamate dysregulation or up-regulation of pro-inflammatory mediators. Supporting this theory is that amantadine, a weak NMDA antagonist, has been reported to alleviate some symptoms of fatigue in multiple sclerosis. Although DA agonism has been attributed to DA agonism, we suggest that its anti-glutamate properties are the etiology of any decrements in fatigue. In a recent study, the degree of fatigue in MS patients was not correlated with systemic markers of inflammatory disease activity such as neopterin (a marker of interferon-γ-activated macrophage activity), serum C-reactive protein, and soluble ICAM-1. We postulate that glutamate receptor density, glutamate dysfunction, abnormal glutamate metabolism or other intracranial inflammatory mediators are major contributors to the etiology of fatigue.

Memantine, administered chronically in oral doses of 5-100 mg/day, advantageously 10-30 mg/day (serum levels ranging from 0.25-2.0 μg/ml) is efficacious in the treatment of fatigue in chronic diseases, chronic fatigue syndromes, and preventing the frontal lobe abnormalities observed on MRI. Memantine administered acutely and chronically in conjunction with current standard medical treatments will be efficacious for the treatment of fatigue. Memantine may administered in conjunction with glycine-site NMDA antagonists, AMPA antagonists or stimulants such as Provigil.

Childbirth

Each childbirth delivery carries a potential risk of an obstetrical complication such as premature labor, prolonged labor, premature rupture of the membranes, abruptio placenta, pelvic-cephalic disproportion, cord around the neck and other hypoxic/anoxic syndromes. These and other syndromes increase the risk of the fetus developing cerebral brain damage and cerebral palsy. Recent reports in neuronal cultures, that NMDA receptor antagonists induce neuronal apoptosis by protein synthesis and caspase-dependent mechanisms, suggests that caution should be used in applying NMDA antagonists to premature neurons. These in vitro experiments revealed a 30-40% neuronal death rate when the NMDA receptor was blocked for 48 hours while the activation of voltage-gated calcium channels attenuated this NMDA antagonist-induced apoptosis. However, the use of cortical cultures, prolonged absolute NMDA receptor blockade without physiological NMDA activity, and the exact relevance of these results to humans is unclear. Specifically, memantine has properties of good placental penetration, minimal teratogenesis, and non-toxic properties to the fetus that make it a valuable prophylactic treatment for all mothers in labor. Memantine has not been associated with birth defects suggesting that it does not produce either a complete or prolonged NMDA channel blockade.

Memantine would be administered most advantageously intravenously or orally for up to 24 hours prior to expected delivery to as a neuroprotectant agent against anoxia, hypoxia, ischemia or mechanical brain trauma. Memantine, administered chronically in oral doses of 5-100 mg/day, advantageously 10-30 mg/day (serum levels ranging from 0.25-2.0 μg/ml) is efficacious in the prophylaxis of brain injury in childbirth. Memantine administered acutely and chronically, as monotherapy or in conjunction with current standard medical treatments will be efficacious for the treatment of complications of childbirth. Memantine will also be used in combination with glycine-site antagonists, AMPA antagonists, calcium channel blockers, anti-oxidants, anti-inflammatory drugs, caspase inhibitors, neurotrophins, or neural stem cell implantation.

Surgical Anesthesia

Each patient that undergoes general anesthesia for any surgical procedure is at risk for hypoxia, anoxia, hypotension, hypoglycemia, spinal cord infarction, and cerebral embolism syndromes (i.e., fat, air). These potential complications place the patient at risk for cerebral or spinal cord damage. An NMDA receptor blocker such as memantine, with neuronal protective properties and an antagonist that allows physiological NMDA activity, is a useful prophylactic treatment prior to anesthesia being administered to prospective patients.

Memantine would be administered most advantageously intravenously or orally at least 24 hours prior to a surgical procedure as a neuroprotectant agent against hypoxia, ischemia or embolism. Memantine, administered chronically in oral doses of 5-100 mg/day, advantageously 10-30 mg/day (serum levels ranging from 0.25-2.0 μg/ml) is efficacious in the prophylaxis of brain injury in surgical procedures. Memantine administered acutely and chronically, as monotherapy or in conjunction with current standard medical treatments will be efficacious for the prevention and treatment of complications of surgical procedures. Memantine will also be used in combination with glycine-site antagonists, AMPA antagonists, calcium channel blockers, anti-oxidants, anti-inflammatory drugs, caspase inhibitors, neurotrophins, or neural stem cell implantation.

Traumatic Brain Injury (TBI)

Blunt trauma to the head has been shown to produce an increase brain glutamate, β-amyloid, inflammatory mediators, cytokine upregulation, and increased CSF levels of quinolinic acid. In addition, the induction of β-amyloid by trauma can produce cerebral neuronal injury and degeneration at lower glutamate concentrations. These neurochemical changes can produce neuronal damage or death by NMDA excitotoxic mechanisms. Brain MRI may reveal hemorrhage, edema or gliosis with either minor or major head trauma. Neuropsychological studies of patients with varying degrees of blunt head trauma reveals long term cognitive abnormalities and psychiatric manifestations. The degree of brain damage is usually determined by the Glasgow Coma Scale but the detection of smaller degrees of TBI require a full scale neuropsychological battery. In animal studies, increased activity of calpains and caspase-3 were found in various brain regions after TBI, suggesting an induction of abnormal intracellular calcium homeostasis. Thus, calpain was increased (30-fold) in the cortex which persisted for up to two weeks in the hippocampus and thalamus. In contrast, no caspase-3 activation was observed in the cortex, while a 2-fold elevation was observed in both the hippocampus and striatum within hours of TBI.

An NMDA receptor blocker such as memantine is a useful treatment for acute head trauma to reduce the acute NMDA-mediated injury, decrease any delayed NMDA apoptosis, and clinically improve any cognitive dysfunction and psychiatric disorders which occur as a sequela to head injury. The duration of treatment with memantine should be at least 2 years, or permanently, since maximum improvement of head injury usually occurs within this time.

Memantine would be administered most advantageously intravenously or orally (via nasogastric tube immediately after head injury) as a neuroprotectant agent against excessive NMDA stimulation due to hypoxia, ischemia or edema. Memantine, administered chronically in oral doses of 5-100 mg/day, advantageously 10-30 mg/day (serum levels ranging from 0.25-2.0 μg/ml) is efficacious in preventing the neurological sequela of head injury including delayed neuronal apoptosis. Memantine administered acutely and chronically, as monotherapy or in conjunction with current standard medical treatments will be efficacious for the treatment of the acute and chronic complications of TBI. Memantine may also be administered with glycine-site NMDA antagonists or AMPA antagonists. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Spinal Cord Injury (SCI)

Excessive NMDA stimulation from glutamate and inflammation contributes to spinal cord injury in acute SCI. The density of NMDA receptors has been reported to be upregulated distal to the site of SCI. We hypothesize that increased NMDA receptor density and function produces neuronal injury, demyelination and degeneration by increasing the level of intracellular calcium and also produces clinical symptoms of spasticity and hyper-reflexia. The NMDA receptor and nerve growth factors neurotrophins (NT-3, BDNF) have inter-dependent actions and abnormal NMDA expression and function may interfere with basal neurotrophin activity. Thus, blockade of the NMDA receptor by an antagonist may allow neurotrophins to regenerate the spinal cord and simultaneously, neurotrophins may improve the function of NMDA receptors. An NMDA receptor blocker such as memantine is a useful treatment of both acute and chronic spinal cord injury. Administration of memantine would reduce the acute NMDA injury, potentially regulate the number and function of NMDA receptors distal to the injury, attenuate demyelination, decrease delayed NMDA apoptosis, clinically improve spasticity and weakness, and allow induction of spinal cord regeneration by nerve growth factors. In animal studies, the concentrations of EAA released upon SCI are neurotoxic to the spinal cord (Lui, 1999). SCI by compression injury results in a rapid primary loss of function and a secondary neurological deficits from an increased QUIN production by inflammatory mechanisms such as activated macrophages (Heyes, 1995). In this study, attenuation of QUIN levels in the spinal cord reduced the magnitude of the neurological deficits.

However, in an animal study of focal spinal cord ischemia, both pre- or posttreatment with memantine (20 mg/kg IP) failed to attenuate the neurological or morphological outcome. This result was attributed to the low receptor affinity of memantine to spinal cord NMDA receptors. These authors concluded that memantine should not be chosen for clinical studies on neuroprotection in spinal cord injuries (von Euler, 1997). We posit that the latter study failed to provide memantine for a sufficient time period to allow for spinal cord regeneration. We have successfully treated a right C3-4 spinal cord transection with a glycine-site NMDA antagonist that was initially administered to decrease spasticity. An unexpected finding was that the patient regained power of the left arm and leg and after six months was able to ambulate without assistance.

Standard medical therapy has been to administer intravenous steroids to acute spinal cord injury. The efficacy of this treatment has been called into question recently and adverse effects such as steroid myopathy have been attributed to this practice. We hypothesize that the administration of steroids to acute SCI will produce increases in glutamate concentrations by decreasing uptake mechanisms. Thus, the concomitant administration of memantine with steroids would allow beneficial effects of steroids while preventing their adverse events.

Memantine would be administered most advantageously intravenously or orally (via nasogastric tube) immediately after diagnosis of SCI, as a neuroprotectant agent against excessive NMDA stimulation. Memantine, administered chronically in oral doses of 5-100 mg/day, advantageously 10-30 mg/day (serum levels ranging from 0.25-2.0 μg/ml) is efficacious in preventing the neurological sequela of spinal cord injury. Memantine administered acutely and chronically, as monotherapy or in conjunction with current standard medical treatments, will be efficacious for the treatment of the acute and chronic complications of SCI. Memantine may also be used in combination with glycine-site NMDA antagonists and AMPA antagonists. In addition memantine may also be combined with future therapies such as calcium channel blockers, partial β and γ secretase inhibitors, anti-oxidants, anti-inflammatory drugs, caspase inhibitors, neurotrophins, or neural stem cell implantation.

Hypoglycemia

Patients who suffer from an acute lowering of the blood sugar are at an increased risk for cerebral brain damage by mechanism involving simulation of the NMDA receptor. Cerebral edema, seizures and permanent cognitive dysfunction are some neurological sequela of hypoglycemia. An NMDA receptor antagonist such as memantine, with neuroprotective properties, is a useful treatment for acute and chronic hypoglycemia. Memantine would reduce the acute NMDA injury, decrease any delayed NMDA apoptosis, and prevent neurological and cognitive sequela of hypoglycemia.

Memantine would be administered most advantageously intravenously or orally (via nasogastric tube) immediately after diagnosis of hypoglycemia as a neuroprotectant agent against excessive NMDA stimulation. Memantine, administered chronically in oral doses of 5-100 mg/day, advantageously 10-30 mg/day (serum levels ranging from 0.25-2.0 μg/ml) is efficacious in preventing the neurological sequela of hypoglycemia. Memantine administered acutely and chronically, as monotherapy or in conjunction with current standard medical treatments will be efficacious for the treatment of the acute and chronic complications of hypoglycemia. Memantine may also be used in combination with glycine-site NMDA antagonists and AMPA antagonists. In addition memantine may also be combined with future therapies such as calcium channel blockers, partial γ and γ secretase inhibitors, anti-oxidants, anti-inflammatory drugs, caspase inhibitors, neurotrophins, or neural stem cell implantation.

Encephalopathy

Encephalopathy is a severe neuropsychiatric syndrome with or without subcortical motor abnormalities usually resulting from metabolic, inflammatory or infectious systemic diseases. An example is hepatic encephalopathy (HE) which occurs in at least cirrhosis, alcohol, primary biliary cirrhosis, sclerosing cholangitis, hepatocellular disease, and Wilson's disease. HE is characterized by systemic conditions that producing secondary neurological symptoms including seizures, cognitive dysfunction, extrapyramidal movements, and dementia. EEG abnormalities occur in one-third of patients with liver failure, cognitive dysfunction occurs in 60% of patients with portacaval shunts while sleep disorders, depression, and anxiety are common symptoms. Cognitive abnormalities in HE correlate with fasting venous blood ammonia. An abnormal uptake and metabolism of ammonia resulting from an increase in blood brain barrier (BBB) permeability also contributes to the syndrome. Brain PET scanning of patients with HE have revealed reductions in cerebral glucose in the anterior cingulate gyrus.

Acute liver failure produces a rapid alteration in mental status, coma and rapid death due to increased intracranial pressure and brainstem herniation from cytotoxic edema. Increases in arterial ammonia concentrations correlate with brainstem edema. PSE (portal-systemic encephalopathy) is associated with chronic liver disease (i.e., cirrhosis and portal hypertension) with symptoms of personality changes, abnormal sleep patterns, asterixis, and coma. In chronic hepatic failure, the neurotoxic concentrations of Mg++ accumulate in the globus pallidus and basal ganglia, producing astrocytic dysfunction and degeneration and possibly abnormal NMDA structure and function. Brain PET (¹³NH3) with mild PSE revealed both an increase in cerebral ammonia metabolic rate and increased permeability of the BBB to ammonia. The brain relies on glutamine synthesis for the removal of excess ammonia and increased ammonia impairs may interfere with post-synaptic inhibition by direct Cl-extrusion and inhibit postsynaptic excitation by a direct effect on glutamate receptor function.

PET scanning studies have revealed decreased rates of both glucose and oxygen utilization in HE that have been attributed to a neuro-transmission failure. Abnormalities in neurotransmission or receptors include the glutamate, GABA, PTBR (peripheral-type benzodiazepine), monoamines (tryptophan, MOA, dopamine, noradrenaline, and histamine), and opiod systems. Ammonia inhibits glutamate uptake which produces an increase in the extracellular concentration of glutamate. A decrease in the densities of binding sites for AMPA/kainate receptors also has been found in HE brains, which produce a relative increase in the number of glutamate receptors. This increase in NMDA receptor density and stimulation may modulate striatal dopamine release and thus produce the clinical motor disturbances. In summary, acute liver failure results in the loss of glutamate transport leading to increased extracellular glutamate and down regulation of AMPA/kainate receptors; while chronic liver impairment have been postulated to produce permanent modifications of the NMDA receptor.

In animals, mild hypothermia has been reported to delay the onset of HE and normalize glutamate transport deficits in acute liver failure, thereby preventing brain swelling and herniation in acute liver failure. In addition, memantine was reported to produce significant improvement in the neurological status of rats with experimental acute liver failure which we attribute to a decrement in the toxic effects of glutamate.

In HE, CSF analysis reveals increased glutamate and quinolinic acid plus other toxic metabolites, which results in multiple clinical neurologic symptoms. We hypothesize that an NMDA receptor antagonist such as memantine is a useful treatment for acute and chronic encephalopathy, including hepatic. Memantine has minimal hepatic toxicity and would reduce NMDA-mediated injury, decrease any delayed NMDA apoptosis, restore neurotransmitter equilibrium, and alleviate the progressive clinical neurological symptoms and cognitive sequela of HE. A prior patent (Lipton U.S. Pat. No. 5,334,618 and U.S. Pat. No. 5,614,560) only mentions hepatic and renal encephalopathy. Those skilled in the art will recognize that we advance the art by expanding the definition of encephalopathy, proposing a disease classification and instruction in the methodology of treatment.

After the initial diagnosis of encephalopathy, from any etiology, memantine would be administered chronically in oral doses of 5-100 mg/day, advantageously 10-30 mg/day (serum levels ranging from 0.25-2.0 μg/ml) for treating the acute and progressive neurological symptoms and sequela of encephalopathy. Encephalopathy will be divided into various diagnostic groups: (1) patients with diseases at risk for encephalopathy, (2) patients with EEG abnormalities consistent with encephalopathy but with no clinical signs or symptoms, (3) acute encephalopathy, and (4) chronic encephalopathy. Memantine will be administered acutely and chronically, as monotherapy or in conjunction with current standard medical treatments. Memantine may also be used in combination with cerebral hypothermia, glycine-site NMDA antagonists and AMPA antagonists. In addition memantine may also be combined with future therapies such as calcium channel blockers, anti-oxidants, anti-inflammatory drugs, caspase inhibitors, calpain inhibitors, neurotrophins, neural stem cell implantation plus potential novel therapies including L-ornithine-L-aspartate and glutamine synthetase inhibitors.

Tumors of the Brain and Spinal Cord and Systemic Malignancies

Brain and spinal cord tumors include primary tumors of glial, neuronal, schwann cell, pinealcyte, meningioma and melanoma, as well as sarcoma, lymphoma and multiple systemic malignancies that metastasize. Gliomas are the most common CNS tumor with GBM, a malignant transformation of astrocytes, a highly malignant invasive and fatal brain tumor (median survival<1 year). Epilepsy was the initial clinical symptom in over 50% of patients and was most common in low-grade astrocytoma (83%) compared to high grade tumors (anaplastic 46% and GBM 36%). CSF analysis has revealed that brain tumors produce elevations in quinolinic acid, an NMDA agonist. We hypothesize that chronic elevations of glutamate, upregulation of cytokines, increased quinolinic acid and inflammatory mediators are involved in the etiology of seizures, brain atrophy, cognitive dysfunction, and neuronal cell death that occurs in patients with CNS neoplasms.

Glioma tumor lines release copious amounts of glutamate and may produce neuronal degeneration by excitotoxicity, clinically intractable tumor-induced seizures, and cell death by apoptosis. The amount of glutamate release was related to the degree of tumor growth suggesting that glutamate is a major factor in glioma malignancy and metastasis. Blockade of the NMDA and AMPA receptor has been reported to decrease the proliferation of multiple tumors types such as colon, adenocarcinoma, astrocytoma and lung carcinoma. The anti-proliferative effect of glutamate antagonists were Ca++ dependent and due to decreased tumor cell division and increased tumor cell death. These observations suggest a direct cytostatic effect of glutamate blockade, possibly by inducing apoptosis in tumor cells by an NMDA mechanism. Glutamate receptor blockers also decreased the motility and invasiveness of tumor cells by converting them to a non-invasive phenotype with fewer psuedopodial protrusions. These antagonists also enhanced the tumoricidal effects of cytostatic drugs, inhibited cell division and migration of tumor cells, accelerated tumor cell death, and altered the morphology of tumor cells in vitro. The efficacy of antiproliferative actions of NMDA antagonists are Ca++ dependent. Elevated Ca++ levels stimulate tumor growth, regulate axon extension and guidance, and influence psuedopodial formation and migration. Tumor cells display immunoreactivity for NR1 and GluR 2/3 subunit membrane proteins. While the exact mechanism of tumor glutamate release is unknown, evidence suggests a prostaglandinE2/chemokine induction or a dysfunction of glutamate receptors. Tumors down-regulate the expression of glutamate transport receptors, providing an additional mechanisms for increasing extracellular glutamate. Thus, simultaneous glutamate and inflammatory blockade may have therapeutic efficacy in treating brain tumors.

Glutamate secreting gliomas may also stimulate local inflammation, facilitate their own metastasis by both paracarine and autocrine mechanisms, induce glutamate release from activated microglia, and alter blood brain barrier permeability to glutamate. We postulate that the resultant elevated glutamate levels may be the etiology of clinically intractable seizures.

Glutamate released by gliomas may produce both a cytotoxic and apoptotic cascade in bordering neuronal tissue and thus create a tract for metastatic invasion, since peritumor brain tissue reveals an inflammatory response with degenerating or necrotic neurons. Direct or indirect microglia activation releases both chemokines and TNF-α, both of which can alter glutamate release from astroglial cells. Stimulation of chemokine receptors on neurons and glia cells triggers both glutamate and TNF-α release, which subsequently produces PG-E2. TNF-α may function in tumor cells by activating caspases, inhibiting glutamate uptake, and producing rapid autocrine/paracrine signaling. Thus, gliomas with high glutamate release have greater brain proliferation and agonist activation of NMDA receptors facilitates tumor expression, possibly by inducing both autocrine and paracrine mechanisms. Both MK-801 and memantine were shown to slow the growth of both brain and systemic glutamate secreting tumors in vitro. In addition, MK-801 attenuated the neuronal loss by neurotoxic concentrations of glutamate suggesting that neuronal death was mediated by NMDA receptor activation.

By utilizing NMDA antagonists, we postulate that down regulation of nuclear messengers produces less apoptosis in surrounding normal peri-tumor tissue. In addition, we hypothesis that systemic, brain and spinal cord tumor cells have an increased receptor density and hyperfunction of NMDA receptors that contributes to both tumor activity and invasivness. Thus, an NMDA antagonist has efficacy both as a direct anti-tumor agent by decreasing the intracellular growth cell signals, decreasing the rate of tumor division and inducing apoptosis of tumor cells. These results suggest that glutamate antagonists possess direct anticancer and anti-tumoricidal activity. Support for this concept is evidence of a calcium microdomain near the NMDA receptor that provides a direct mechanism for a synapse to nucleus signaling. Thus, stimulation of extracellular signal-regulated kinase (ERK_{1/2}) produces nuclear signaling, stimulates SRE-dependent gene expression and prolongs the CREB-mediated gene expression, all independent of global increases in cellular Ca++ concentration. Thus, NMDA activation of the ERK_{1/2} pathway results in propagation of extracellular synaptic signals in a Ca++ dependent manner to the nucleus. While the relationship of the NMDA receptor to the EGFR (epidermal growth factor receptor) is unknown, down regulation or decreased function of the EGFR in brain tumors by glutamate antagonism by calcium mechanisms would simulate an anti-tumor treatment and decrease the degree and severity of metastasis, similarly to the recent anticancer EGFR blockers. We hypothesize that glutamate receptors on all tumor cells, by abnormal number/structure or function, could contribute to regulation of proliferation and migration of tumor cells by both autocrine and paracrine mechanisms via a Ca++ mechanism.

In summary, an NMDA antagonist would also decrease the frequency of seizures, brain atrophy, reduce apoptosis of normal neuronal cells, and decrease neuronal necrosis. Memantine was shown to decrease in vitro proliferation of tumor cells including lung, rhabdomyosarcoma, medulloblastoma, and thyroid adenocarcinoma. Tumor expansion in animals facilitated by glutamate secretion was blocked by memantine (25 mg/kg IP) by memantine. Tumor volume was decreased by 25% and in vitro proliferation was decreased in vitro at levels of 100 μM/ml, which are unobtainable at therapeutic human doses. Thus, we propose alternative mechanisms of tumor inhibition such as NMDA induction of nuclear messengers that decreases cell division, NMDA induction of tumor apoptosis, or an interaction with other intracellular pathways. The recent demonstration of a serological marker TAA (tumor-associated antigens) that is reliable for the early detection of cancer and sensitive and specific for the detection of multiple cancers as well as assessing the progress and recurrence of cancer, suggests that early treatment with memantine may be warranted.

Memantine would be administered most advantageously intravenously or orally after the initial diagnosis of primary or metastatic brain, spinal cord tumors or systemic tumors. Memantine would be administered for the purpose of a direct tumoricidal agent, as an anti-metastasis agent, and a neuroprotectant agent to neutralize the effects of excessive NMDA stimulation. In addition, memantine administered prior to brain radiation, steroid treatment, or chemotherapy treatment for CNS tumors would decrease the side effects of these therapies while increasing the efficacy of these therapies. We hypothesize mechanisms of decreasing tumor glutamate release and thereby decreasing seizures, limiting tumor metastasis, and inducing tumor apoptosis by NMDA induction of cell nuclear activity.

Memantine, administered chronically in intravenous or oral doses of 5-100 mg/day, advantageously 10-30 mg/day (serum levels ranging from 0.25-2.0 μg/ml) is efficacious in controlling the progressive neurological symptoms and sequela of systemic, brain and spinal cord tumors. Memantine administered acutely and chronically, in conjunction with current standard medical treatments (i.e., cytostatic) for systemic, brain and spinal cord tumors will be efficacious for the treatment of the acute and chronic neurological complications of brain and spinal cord tumors. Memantine will also be co-administered with other calcium channel blockers (L-, N- and others) glycine-site NMDA blockers, AMPA blockers, glutamate synthesis inhibitors, inhibition of glutamate resynthesis or the precursor glutamine, NOS inhibitors, inhibition of glutamine synthetase, or stimulation of neuronal or astroglial transporters. Memantine will also be co-administered with EGFR antagonists, monoclonal antibodies, and other tumor receptor antibodies or intracellular enzyme blockers. Memantine will also be used in combination with anti-oxidants, anti-inflammatory drugs, caspase inhibitors, neurotrophins, or neural stem cell implantation.

Cerebellar Degeneration and Ataxias

Cerebellar degeneration may be classified as primary (familial and genetic) or secondary to systemic disorders (myxedema, alcoholic). Diseases include at least Friedrich's ataxia, cerebellar cortical ataxia, spinocerebellar disease, and ataxias complicated with brainstem and other neurological disorders (dentatorubral degeneration, autosomal dominant ataxias). Clinical symptoms include at least ataxia, dysmetria, intention tremor, hypotonia, dysarthria, and nystagmus. Cerebellar degenerations may be classified as: (1) acute: intoxication (lithium, dilantin), toxins (mercury), or post-infectious; (2) subacute: tumors, alcohol, nutritional, paraneoplastic; or (3) chronic progressive ataxia which may be hereditary: Friedrich ataxia (early), cerebellar cortical ataxias, and complicated cerebellar ataxia (which includes OPCA or olivopontocerebellar degeneration). Those skilled in the art will recognize that OPCA is an older term usually used to define hereditary cerebellar-pontine atrophy and was a descriptive term for atrophy of the portions of the brainstem and cerebellum. Friedrich ataxia, a mutation of chromosome 9, is a trinucleotide GAA repeat error that codes for the protein “frataxin”. A current hypothesis is that frataxin is a mitochondrial associated protein that produces cellular dysfunction and a failure of energy metabolism. Decreased mitochondrial energy decrease the resting membrane potential, unlocks the Mg++ gating mechanism, and causes NMDA receptor overstimulation and neuronal death. The subunit composition of cerebellar NMDA receptors differs from that of the cortex, predominantly being NR2C and NR2D. Thus, cerebellar degeneration may be induced by a predominant NMDA mediated mechanism. The preferential blocking of memantine to the NR2C and NR2D subunits predicts a strong therapeutic response. While a prior patent (Lipton U.S. Pat. No. 5,614,560) mentions OPCA, our patent expands and clarifies this outdated definition of progressive ataxia.

Memantine would be administered orally after the diagnosis of cerebellar degeneration as a neuroprotectant agent against potential excessive NR2C and NR2D NMDA stimulation. Memantine, administered chronically in oral doses of 5-100 mg/day, advantageously 10-30 mg/day (serum levels ranging from 0.25-2.0 μg/ml) is efficacious in controlling the acute and chronic progressive neurological symptoms and sequela of cerebellar degeneration. Memantine administered intravenously for acute cerebellar degenerations, or orally for subacute and chronic types of cerebellar degenerations, in conjunction with current standard medical treatments (i.e., 5-hydroxytryptophan), will be efficacious for the treatment of the acute and chronic neurological complications of cerebellar degeneration. Memantine may also be used in combination with glycine-site NMDA antagonists and AMPA antagonists. In addition memantine may also be combined with future therapies such as calcium channel blockers, anti-oxidants, anti-inflammatory drugs, caspases, calpain inhibitors, neurotrophins, or neural stem cell implantation.

Pre-Clinical Huntington's Disease

Huntington's Disease, also referred to as HD, is an autosomal dominant disease, caused by a mutation in chromosome 4, which produces a mutation of the protein huntingtin. The function of huntington is to regulate the production of another protein, BDNF (brain-derived neurotropohic factor) that is essential for survival of striatal neurons. A more recent hypothesis implicates an interactive role between NMDA receptors and BDNF in the etiology and clinical expression of HD. Thus, the finding that memantine increased BDNF mRNA levels in the rat limbic system and also induced isoforms of this receptor (trkB) suggest a potential neuroprotective effect in the treatment of preclinical HD. In HD, striatal neurons selectively die in the brain and patients with HD do not have elevated CSF or brain parenchymal QUIN acid concentrations. However, presymptomatic patients have been found to have a 50% reduction in the number of NMDA receptors. Thus, normal levels of QUIN may be overstimulating an insufficient number of NMDA receptors contributing to neuronal cell death.

We propose that presymptomatic at-risk HD patients be diagnosed on the basis of PET scanning using either labeled memantine or felbamate. Patients known to have a decrease in the density of NMDA receptors would then be treated prophylactically with oral memantine to decrease functional quinolinic and glutamate toxicity, and prevent neural apoptosis. A prior patent (Lipton 1997) mentions HD but not patients at risk. Those skilled in the art will realized that prophylactic treatment of asymptomatic patients differs from treating a clinical disease.

Memantine will be administered orally and chronically in patients with HD who are at risk but asymptomatic, as monotherapy, or in conjunction with other treatments or medications that attenuate glutamate or block glutamate receptors. Memantine, administered chronically in oral doses of 5-100 mg/day, advantageously 10-30 mg/day (serum levels ranging from 0.25-2.0 μg/ml) is efficacious in the treatment of the clinical symptoms of HD. Memantine may also be used in combination with glycine-site NMDA antagonists, AMPA antagonists, calcium channel blockers, anti-oxidants, anti-inflammatory drugs, caspases, calpain inhibitors, neurotrophins (NT3, BDNF), or neural stem cell implantation.

Depression

Depression is defined as a neurochemical brain disorder in which a disturbance of mood is either a primary determinant or constitutes the core manifestation. Symptoms include a state of morbid sadness, dejection, or melancholy with a decrease in functional activity. Vegetative depression is clinically expressed as low mood, excessive somnolence and obesity. Secondary depression may be defined as an affective disorder caused by a systematic or neurological disease. Common neurological diseases which are complicated by depression include Parkinson's disease, head trauma, brain tumors, stroke, dementia, and sleep apnea. Common systemic diseases include infections, endocrine disorders, collagen vascular diseases, nutritional deficiencies and neoplastic diseases. For example, secondary depression is present in up to 50% of post-myocardial infarction patients and causes a three fold increase in mortality than in non-depressed patients with myocardial infarction.

We hypothesize that glutamate dysregulation may be involved in the etiology of primary depression (unipolar, bipolar etc.) and interact with the serotoninergic system. We further hypothesize that upregulation of cytokines, quinolinic acid etc. may contribute to the clinical expression of secondary depression, apathy and fatigue. The effect of memantine on serotonin levels, a major transmitter in depression, is unknown. I have successfully treated a patient with a long history of bipolar depression with an NMDA antagonist. An unexpected finding was that efficacy was observed with both monotherapy and in combination with lower doses of Prozac.

Memantine would be administered orally and chronically in patients diagnosed primary or secondary depression. Memantine, administered chronically in oral doses of 5-100 mg/day, advantageously 10-30 mg/day (serum levels ranging from 0.25-2.0 μg/ml) is efficacious in decreasing the severity of depression and reducing the morbidity and mortality of depression of chronic diseases, when combined with other standard treatments of depression. Memantine, when administered in conjunction with current standard medical treatments (i.e., SSRI or selective serotonin uptake inhibitors) and glycine-site NMDA antagonists will be efficacious in both primary and secondary depression.

Neuroprotection in Patients with Cerebrovascular Risk Factors

Cerebrovascular disease is a common illness linked to risk factor(s) such as hypertension, hyperlipidemia, cardiac disease, smoking, and diabetes while the prevalence of stroke has declined with more effective treatment of these medical conditions. We define vascular dementia as a chronic progressive cognitive decline in patients with cerebrovascular risk factors (CVRF's) which cause excessive and chronic overstimulation of both NMDA and non-NMDA receptors by the chronic increase in glutamate, inflammatory mediators, cytokines, quinolinic acid, and other toxic mediators that produces neuronal cell dysfunction, necrosis or premature apoptosis. This definition implies an absence of hypoperfusion, ischemia, hypoxia or anoxia and implies a “neurochemical etiology of neurotoxicity, necrose and leukoariosis”. In patients with CVRF's, plasma homocysteine levels can be increased and are correlated with dementia. Homocysteic acid, a metabolite of homocysteine, can cause neuronal excitoxicity by stimulation f the NMDA receptor producing brain damage. Thus, in a subset of patients with the single risk factor of medically controlled chronic hypertension for at least ten years, abnormal brain imaging, brain metabolism (by PET scanning) and cognitive dysfunction was observed. The latter patients were monitored to exclude for ischemia and hypoxia as an etiologic factor. With chronic risk factors, eventually such patients may subsequently have various superimposed acute or chronic strokes syndromes such as lacunar infarcts, hemorrhagic or ischemic strokes, chronic ischemia that would produce additional cognitive dysfunction. We hypothesize that various CVRF's causes vascular endothelial damage, and upregulates cytokines, inflammatory mediators, glutamate, nitric oxide, and other NMDA toxins. These neurotoxins cause chronic overstimulation of the NMDA receptor resulting in neuronal dysfunction and eventually neuronal cell death in the absence of hypoperfusion, hypoxia or anoxia. We further hypothesize that this chronic neurochemical process or “necrotoxosis” subsequently results in cognitive dysfunction and the brain abnormalities observed on neuroimaging such as atrophy and leukoariosis.

Memantine may function by attenuating the neuronal depolarization, removal of the Mg++ block, and excessive non-NMDA and NMDA stimulation by these necrotoxic mediators resulting in an attenuation of necrosis and apoptosis. Memantine will also simultaneously potentiate LTP and improve cognition in these patients.

Memantine would be administered orally and prophylactically in all patients with CVRF. Memantine, administered chronically in oral doses of 5-100 mg/day, advantageously 10-30 mg/day (serum levels ranging from 0.25-2.0 μg/ml) is efficacious in decreasing the severity of both morbidity and mortality of complications of CVRF. Memantine will be administered concomitantly with other standard medications for treating cerebrovascular risk factors such as hypertension, increased cholesterol, diabetes etc. Memantine will also be co-administered with glycine-site NMDA antagonists, AMPA antagonists, calcium channel blockers, anti-oxidants, anti-inflammatory drugs, caspase inhibitiors, calpain inhibitors, neurotrophins (NT3, BDNF), or neural stem cell implantation, and will be efficacious in preventing and reducing necrosis, apoptosis and future cerebrovascular accidents.

Neuroprotection in Post-ischemic Neurovascular Syndromes

Neurovascular syndromes include at least TIA, amourosis fugax, cerebral hemorrhage, cerebral infarction (ischemic and thrombotic) and lacunar syndromes. Since most ischemic syndromes are acute and cause neuronal injury immediately, we define post-ischemic neurovascular syndromes within a time frame longer than three days, since the ischemic process has abated and only neuronal injury remains. Current therapeutic clinical trials have concentrated on the attempt to prevent neuronal damage only in the acute setting, with the duration of treatment being minimal compared to the time when the end point of the clinical trial is actually measured. Thus, a recent study concluded that glycine-site NMDA antagonists were ineffective in treating acute stroke syndromes when treatment was limited to three days post-ischemic event. However, using quantitative autoradiography, prolonged alterations in NMDA, AMPA, and KA receptor density were noted following photothrombotic ischemic lesions in the rat. Increases in the binding density of NMDA receptors were observed in both hemispheres for up to 30 days. In the contralateral hemisphere, increased NMDA receptors occurred within 4 hours whereas it appeared after a delay of 14 days ipsilaterally. AMPA and KA receptor binding density were unchanged. These results suggests that the cellular translational process is differentially regulated by the phenomena of spreading depression. The delayed up-regulation of ipsilateral NMDA receptor binding may be due to a translational block similar to that previously described for GABAₐ receptor subunits. Thus cortical disinhibition was found to be widespread after focal photothrombotic lesion and was associated with an alteration of the balance between excitatory and inhibitory amino acid receptors in the cerebral ischemic lesion. After 4 hours of ischemia, binding density had decreased in the center of the lesioned area: NMDA (−60%), AMPA (−54%), and KA (−13%) while after the second week, binding density was NMDA (−87%), AMPA (−71%), and KA (−80%). In histological intact areas (exofocal areas) in the ipsilateral hemisphere, NMDA receptors increased 8% by day 14 in the primary motor cortex and significantly increased at 30 days in both the primary motor cortex (12%) and primary somatosensory cortex (15%). The increase in the density of NMDA receptors in the contralateral hemisphere occurred earlier than the ipsilateral hemisphere. NMDA receptor density was altered: upregulated at hour 4, primary motor cortex (+15%) by day 3, and primary somatosensory cortex (+18%), decreased at the end of the second week, and increased at 30 day in motor (+16%) and sensory (+18%) cortex. In conclusion, this study revealed significant but transient up-regulation of NMDA receptors in remote cortical areas of the contralateral hemisphere within 3 days, an increase in NMDA binding density in both hemispheres at 30 days, NMDA receptor changes that correlated with the widespread hyperexcitability responses of post-synaptic potentials. In remote areas bilaterally, a decrease in the density of binding GABAₐ receptor binding occurred with an increase in NMDA receptor density. The imbalance of excitatory-inhibitory receptors was associated with cortical dysfunction in the intact remote areas, and while MK-801 was able to inhibit cortical spreading after infarction, it was unable to reverse the hyperexcitability. In the hemisphere ipsilateral to the lesion, NMDA receptors increase after 2 weeks, while the photothrombotic lesions produced spreading depressions in the ipsilateral hemisphere, induction of IEG (immediate early genes) and neurotrophic factors, and astrocytic activation. This study concluded that the delay in the up-regulation of NMDA receptors in the ipsilateral hemisphere was due to a translational block by cortical spreading depressions. Based on these observations of delayed abnormal NMDA function, we hypothesize that it is unlikely that acute short-term doses of NMDA antagonists at the onset of any ischemic lesion will have a significant long term effect on clinical outcome.

Unilateral, permanent MCA occlusion in exofocal neocortical areas of the mouse was shown to produce long term excitability. Quantitative in vitro autoradiography for NMDA, AMPA, KA, and GABA receptors revealed that all of these binding sites were severely reduced in the core of the ischemic lesion. GABA binding sites were significantly decreased 4 weeks after ischemia in the motor cortex (layer V and VI), NMDA binding sites were increased in these areas in layer III and IV, while AMPA/KA sites were not significantly increased. However, all binding sites were also reduced in the retrograde affected portions of the ipsilateral thalamic nucleus (VPN). Thus, permanent local ischemia leads to a long-term and widespread imbalance between the binding sites of excitatory and inhibitory receptors in neocortical areas distal from the focus of post-ischemic tissue damage. Upregulation of NMDA binding sites (in primary somatosensory cortex and layer III of the frontal cortex) and down-regulation of GABA binding sites occurred in the ipsi- and contralateral neocortex. These receptor abnormalities are the etiology of the cortical hyperexcitability with epileptiform field potentials and the long duration of excitatory post-synaptic potentials observed 4 weeks after ischemia. Neuronal reduction and severe gliosis in the ipsilateral but not contralateral thalamus (VPL and VPM) suggest that the cortical lesions can induce both a retrograde neuronal damage and severe gliosis in specific thalamic relay nuclei. Conversely, AMPA receptors showed an ipsilateral increase in the VPM thalamic nucleus by 22%. Receptor density quantization revealed an elevated NMDA (ipsilateral +26% and contralateral +23%) and a decrease in GABA receptors (ipsilateral −21% and contralateral −22%). Thus, receptor imbalance occurred in remote, histologically normal neocortical areas of the contralateral hemisphere.

CSF analysis in patients with acute middle cerebral artery stroke (<8 hours) have revealed significant elevations of aspartate, glutamic acid, glycine and alanine. In addition, CSF levels of nitrite (a metabolite of nitric oxide) and its precursor arginine were also significantly higher. The correlation of CSF arginine and nitrite with glutamic acid suggest that these neurotoxic mediators contribute to acute neuronal death in stroke patients. The total duration of these CSF changes in stroke patients remain to be elucidated.

Most recent clinical studies that have utilized NMDA receptor therapy in acute clinical stroke trials have utilized a limited time frame, usually less than a week, which may be insufficient to show long term efficacy. We propose that the above data strongly suggests that NMDA and glutamate abnormalities may last months in the absence of chronic treatment and therefore endpoints in such trials require chronic treatment for months or even permanently. I have treated a patient with vascular dementia from leukoariosis from hypertension, lacunar infarcts, and a intracerebral hemorrhage with a glycine-site NMDA receptor for a duration of 4 months. An unexpected finding was global improvement in all cognitive tests at 6 months with improved activities of daily living persisting for years.

Memantine administered IV acutely in patients with TIA and then chronically by an oral route will attenuate the degree of cerebral neuronal damage and decrease future episodes of TIA and stroke. Memantine administered IV immediately at the onset of acute stroke and then prophylactically and chronically, or permanently, by an oral route will attenuate the degree of abnormal NMDA receptor density, decrease cortical spreading depression, decrease cerebral neuronal damage by apoptosis or necrosis, and decrease the incidence of post-stroke seizures. Memantine administered chronically by an oral route in all chronic post-stroke patients will attenuate delayed cellular necrosis and apoptosis and assist in neuronal plasticity during the cerebral regenerative phase. Memantine, administered acutely by the intravenous route or chronically in oral doses of 5-100 mg/day, advantageously 10-30 mg/day (serum levels ranging from 0.25-2.0 μg/ml) is efficacious in the treatment of cerebrovascular diseases and stroke syndromes. Memantine may be administered concomitantly with current standard medical treatments as well as in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or neural stem cell implantation. The duration of memantine treatment after an ischemic or stroke-like syndrome should be indefinitely.

Migraine

With the recent identification of the brain-specific P/Q-type Ca++ channel gene CACNA1A contributing to the pathogenesis of migraine, a strong but complex genetic component contributing to dysregulation of neuronal calcium homeostasis has been implicated. In addition, plasma glutamate and aspartate measurements in migraine patients with and without aura, between and during attacks, have been reported to be abnormal. Between attacks, migraineurs (>with aura) had a substantially higher glutamate and aspartate levels, with additional increases during a migraine attack. These results suggest a defective cellular reuptake mechanism in migraine at the neuronal/glial level that predisposes the brain to develop spreading cortical depression (SCD). Glutamate has been implicated in migraine pathogenesis: (1) NMDA receptor activation contributes to initiation, propagation, and duration of SD; (2) brain energy metabolism is altered in migraine patients; and (3) brain Mg++ concentrations decrease during migraine attacks resulting in enhanced NMDA receptor sensitivity and decreases in the threshold to SCD. Finally, excessive oral intake of glutamate or domoic acid (>40%) can induce symptoms of severe headache. MRI studies of brains in migraine patients revealed an increase in subcortical gliosis in classical>common migraine which we hypothesize is due to elevated glutamate levels inducing apoptosis or chronic transient ischemia due to SCD.

Based on evidence from various neuroimaging techniques, migraine has been hypothesized to be of primary neurogenic etiology. fMRI has revealed that a headache is preceded by neuronal suppression that originates in the occipital cortex and slowly propagates anteriorly. The neuronal suppression was accompanied by vasodilation and tissue hyperoxygenation, similar to the phenomena of SCD. Utilizing perfusion MRI, a reduction in CBF (cerebral blood flow) was observed in the occipital cortex contralateral to the visual defect during migraine with aura (MwA) but not with migraine without aura (Mw/oA), again supporting the concept of SCD. However, diffusion-weighted MRI techniques sensitive to ischemia revealed no alterations in CBF and normal neuronal osmotic gradients, suggesting that MwA is not an ischemic event. Notably, both PET and MRI studies have revealed brainstem activation (dorsal raphe, periaqueductal gray, locus ceruleus, red nucleus and substantia nigra) in spontaneous migraine attacks. PET and SPECT scan studies have also revealed decreased CBF with Mw/oA compared to the interictal period but these decrements did not approach ischemic values. Finally, using MEG (magnetoencephalography), the occipital cortex was found to be neuronally hyperexcitable. This result provides additional evidence for a triggering of SCD and induction of migraine aura, again supporting a primary neural basis of migraine. Studies with MRS (spectroscopy) have revealed abnormal cerebral metabolism (in the absence of an alteration if pH) during a headache after an aura. Finally, in a patient studied five weeks after a MwA, abnormal oxidative impairment was still documented.

In summary, evidence against cortical ischemia in migraine attacks include: (1) an insufficient magnitude of CBF decrements to produce significant ischemia, (2) normal DW-MRI suggesting no ischemic-mediated neuronal injury, (3) headache pain preceding hyperemia suggesting that the pain is generated by mechanical distension of nociceptive neurons in dilated vessel walls, (4) evidence that the aura is generated by SCD with transient neuronal dysfunction and secondary decreases in regional CBF, and (5) PET scans revealing activation of midbrain and pons in migraine and hypothalamic grey areas in cluster headache providing data that pain can be derived from the neural innervation of the cranial circulation. Vasodilation of the major arteries during acute headache pain has been attributed to the activation of neural vasodilator mechanisms.

While memantine has an adverse profile of headache in up to 10% of patients who do not suffer from migraine, this side effect is mild and usually transitory and dose related. Memantine administered to patients with various subtypes of migraine will decrease the formation of SCD by NMDA receptor mechanisms and attenuate additional neurogenic contributions to pain. I have treated a patient with intractable migraine that required constant ER admission for Demerol IM with a glycine-site NMDA antagonist. An unexpected finding was total control of her migraine headaches for years on chronic oral doses without adverse events.

Memantine administered IV acutely in patients with intractable migraine headaches and then chronically by an oral route will attenuate the degree of SCD and neurogenic hyperexcitability. Memantine administered prophylactically and then chronically, or permanently, by an oral route will attenuate abnormal NMDA receptor function, decrease SCD, attenuate the degree of subcortical gliosis, and decrease the clinical severity of migraine. Memantine administered chronically by an oral route in all migraine syndromes by the intravenous route or chronically in oral doses of 5-100 mg/day, advantageously 10-30 mg/day (serum levels ranging from 0.25-2.0 μg/ml) is efficacious in the treatment of migraine syndromes. Memantine may be administered concomitantly with current standard medical treatments and in combination with glycine-site NMDA antagonists, AMPA antagonists, calcium channel blockers, anti-oxidants, anti-inflammatory drugs, caspase inhibitors, calpain inhibitors, and neurotrophins (NT3, BDNF). The duration of memantine treatment will be determined clinically but may be indefinitely.

Vertigo and Vestibular Symptoms

The vestibular system is an integrative sensimotor complex that integrates the sensation of head movement with the generation of vestibular-ocular reflexes (VOR) for stabilizing gaze and vestibulo-spinal reflexes (VSR) for controlling body posture. The central vestibular neurons receive ipsilateral sensory inputs, polysynaptic visual and proprioceptive impulses, plus projections from the cortical, cerebellar and spinal cord tracts.

Peripheral vestibular system damage, either receptor or nerve damage, produces a syndrome of ocular motor and postural disorders due to disruption of these vestibular-ocular and vestibular-spinal pathways. UVD (unilateral vestibular deafferentiation) will produce a severe acute imbalance in neuronal activity between ipsilateral and contralateral vestibular nerve complexes (VNC). Vestibular compensation, such as spontaneous ocular nystagmus (SON) occurs within days or weeks, while dynamic symptoms such as abnormal sensitivity of VNC to head movements (VOR and VSR) are incomplete, requiring a longer duration due to neuronal plasticity within the CNS. After UVD, long-lasting or permanent deficits in VOR results in oscillopsia, while both OKR (optokinetic reflexes) and OKR after nystagmus are decreased. In contrast, patients with idiopathic vestibular failure in the absence of spontaneous nystagmus, experience deficits in object motion perception even when the head is stationary. The resolution of oscillopsia following UVD has been postulated to be due to a visual system compensation that will effectively ‘null out’ abnormal amounts of retinal slip. UVD may also cause a disruption of spatial memory process, possibly due to vestibular inputs to the hippocampus, limbic system, and neocortex. Thus, the recovery of resting activity in the ipsilateral VNC is an important event in the compensation of static symptoms, involving alterations in cerebellar function and input. A substantial part of neuronal recovery in ipsilateral VNC has been shown to occur within the first 10 hours post-UVD.

Accumulating evidence supports the role of NMDA receptors, NO signaling, neurotrophic expression and phosphorylation both in the VNC and cerebellar flocculus during the process of both vestibular compensation and static symptoms of UVD. NMDA receptor antagonist injection following UVD has been shown to alter this vestibular compensation process. Transient increases in NMDA expression of NR1 and NR2C receptor subunits occur in ipsilateral MVN (medial vestibular nucleus) suggesting that NMDA receptors reorganization is an early and important event. Importantly, NMDA (MK-801) antagonist injected directly into the VNC prior to UVD, reduced the severity of the vestibular syndrome especially SON and YHT (yaw head tilt). As well, metabotropic (mGLu) receptor antagonists injected into the ipsilateral VNC resulted in large decreases in SON frequency and YHT during the first 50 hours of compensation, suggesting mGLu receptors also play an important role in vestibular compensation. Taken together, these results suggest that at least both NMDA and mGLu receptor function are involved in the LTP and LTD of the VNC. The compensation mechanism also involves modification of existing proteins (i.e., phosphorylation) since UVD has been shown to produce a bilateral increase in protein-kinase C (PKC). Neurotrophic factors are also contributory since NT4 knockout mice exhibited a delay in compensation while an increase in high affinity neurotrophin receptors has been observed after UVD. It is also known that the stress response to UVD is also critically important, since dexamethasone increased the rate of the development of vestibular compensation and regulates neuronal plasticity in ipsilateral VNC following UVD. NOS (nitric oxide synthetase) inhibitors produce a delayed vestibular compensation, with an altered NOS expression in the cerebellar flocculus and decreased NOS in the ipsilateral MVN for up to 50 hours. In summary, the compensation of the static ocular motor and postural symptoms occurs rapidly and completely, while dynamic compensation is both incomplete and requires a longer duration. Static compensation appears associated with substantial recovery of the resting activity in the ipsilateral VNC.

While controversial, the cerebellar cortex has been implicated in the visual-vestibular adaptation and in vestibular compensation. The VOR adaptation is associated with both plasticity at the cerebellar cortex and vestibular nuclei. During vestibular compensation, there is a return of resting discharge activity in the VN ipsilateral to the lesion that reflects a change in sensitivity of these neurons. This form of post-lesional plasticity includes multiple mechanisms: upregulation of NMDA receptors within the ipsilateral MVN, amplification of intrinsic membrane properties, and an ipsilateral down regulation and contra-lateral upregulation of GABA post-synaptic receptors. All of these latter membrane and receptor changes require the activation of glucocorticoid receptors.

Glutamate is integral to both LTP and LTD processes of synaptic plasticity in the vestibular nucleus. The NMDA receptor is the main component in this plasticity process but is modulated by both AMPA and mGLu receptors. Thus, the processes of LTP and LTD in the MVN, induction and maintenance of vestibular compensation and permanent vestibular phenomena are NMDA receptor dependent. The most common and abundant NMDA receptor subunits in the MVN are NMDA R12C (low Mg++ sensitivity) and R12A (high Mg++ sensitivity). The dorsal portion of the MVN also appears to function in LTD by an enhanced release of GABA from potentiated interneurons. Both mGLu-receptors and PAF (platelet activating factor) increase glutamate release, activate post-synaptic NMDA receptors and possibly act as a retrograde messengers. NO is also released in the vestibular nuclei in the compensated state following UL and may also function as a retrograde messenger. NMDA receptor activation has been shown to influence both c-fos expression in VNC and NOS expression in the cerebellum suggesting that the MVN has synaptic mechanisms that contribute to the formation of vestibular LTP.

Taken together, the above results suggest a pivotal role for both glutamate and NMDA receptors in producing the clinical symptoms of vertigo, in generating the acute and chronic compensatory mechanisms, and inducing regenerative neuronal plasticity. Thus, since memantine has preferential interaction at the NR2C subunit of the NMDA receptor, we predict therapeutic efficacy in the treatment of both acute vertigo and dysequilibrium syndromes, as well as acute and chronic vestibular syndromes.

Memantine administered intravenously or orally and chronically in patients with various forms of vertigo, dysequilibrium and vestibular syndromes, as monotherapy or in conjunction with other standard treatments, would provide both efficacy and safety in these conditions. Memantine would be administered concomitantly and prophylactically in patients at risk for these disorders. Memantine, administered chronically in oral doses of 5-100 mg/day, advantageously 10-30 mg/day (serum levels ranging from 0.25-2.0 g/ml) is efficacious in the treatment of vertigo and vestibular syndromes by reducing neuronal necrosis or apoptosis as well as inducing compensatory neuronal plasticity in such patients. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or neural stem cell implantation.

Tinnitus and Cochlear Disorders

Tinnitus is defined as a subjective or phantom auditory perception of sound in the absence of an appropriate external stimulus. Tinnitus is a symptom of multiple etiologies, not a specific disease, and is initiated by processes that damage the cochlea (noise, viral infection, and ototoxic damage). Hyperacusis, a state of an hyper-acute sense of hearing, is a pretinnitus state and a manifestation of increased central auditory gain. Tinnitus is generated within the auditory system from either peripheral or central origins, but is clinically more severe when associated with cochlear pathology. While generation is postulated to occur with discordant damage or function of the outer and inner hair cell systems, the final stage of tinnitus emergence is the perception, evaluation and dysfunction of the cortical association areas and the limbic system. The strong imprinting of the tinnitus sound in the CNS, once the abnormal pattern of neural activity is detected and classified by the brain, can be chronically persistent and eventually associated with neuropyschiatric manifestations.

In animal models, observed changes in the spontaneous activity of single neurons in the inferior colliculus (IC) are consistent with increased abnormal neuronal activity within auditory pathways. These abnormalities are similar to conditions known to produce tinnitus in humans. The IC, which contains NMDA receptors, is postulated to be the primary anatomical location in the ascending auditory pathway where noise-induced neuronal plasticity occurs, resulting in altered neuronal processing of auditory information. Noise (or tone) exposure produces acute and chronic alterations in the excitability of the IC. In animal models, spontaneous activity of single units recorded from the IC, before and after salicylate-administration, increased the mean rate of spontaneous discharges and also produced abnormal, epileptic-like, neuronal activity that involved both GABA and calcium currents. The persistence of tinnitus in patients even after VIII (cochlear nerve) transection provides evidence for the central neuronal origin of tinnitus and implies that auditory cortical plasticity is a major factor in the generation of tinnitus.

The process of tinnitus is hypothesized to originate with a sensineural hearing loss in the auditory periphery such as a cochlear lesion, loud noise exposure, or age-related hair cell loss. The resulting abnormal neuronal activity arising from the auditory pathway is then interpreted as sound at the cortical level and produces a cortical reorganization or alteration in neuronal plasticity. Support for this hypothesis is provided by PET scanning in which phantom auditory sensation(s) increase regional cerebral blood flow in both tempero-parietal association areas, but not in the primary auditory cortex. Therefore, the perceptual qualities of clinical tinnitus (intensity, frequency, spatial localization) originate in the tempero-parietal regions of the brain. Abnormal interactions between the limbic and auditory system by brain PET scanning imply that the generation of tinnitus is due to cortical processing of ascending subcortical auditory signals that subsequently induce cortical plasticity.

While glutamate mediates neurotransmission between inner hair (IH) and afferent auditory neurons, both NMDA and AMPA receptors are present on afferent neurons of IH cells in the mammalian cochlea. Elevations of quinolinic acid are found in inner ear effusions that produce cochlear hearing loss in inflammatory processes of the middle ear. We hypothesize that neurotoxicity induced by excessive glutamate release has a crucial role in cochlear pathology, such as ischemia, noise trauma, head trauma, presbyacusis, Meniere's disease, sudden hearing loss, pure neurosensory deafness, hereditary hearing loss with retinal disease, and hereditary hearing loss with system atrophies of the nervous system. Inner ear diseases, hearing loss and tinnitus may be triggered by an excessive influx of Ca++ into post-synaptic dendrites of IHC afferents through ionotropic glutamate channels, suggesting a critical role for calcium homeostasis in the generation of tinnitus. Additionally, cochlear ototoxicity such as hearing loss and deafness occurs in 20-33% of patients who utilize aminoglycoside antibiotics. These disorders are dose-dependent, usually permanent, and closely parallel the destruction of cochlear hair cells and later, the spiral ganglion. A postulated mechanism is agonist stimulation at the polyamine site of the NMDA receptor producing glutamate excitotoxicity. Concurrent administration of a either a competitive antagonist or a polyamine antagonist of the NMDA receptor attenuated both the hearing loss and destruction of the cochlear hair cells.

The efficacy of current therapy for tinnitus is controversial but includes devices that mask tinnitus and TRT (tinnitus retraining therapy) which is a technique utilizing white noise for a period of time to assist the patient to habituate to their tinnitus. In animal models, memantine has been shown to selectively inhibit the NMDA stimulated activity of induced activity of inner hair cell afferents. We postulate that the administration of memantine would attenuate the neurotoxicity mechanisms of cochlear disorders and tinnitus, and also attenuate any abnormal neuronal plasticity in the temporal-parietal association cortex resulting in clinical efficacy for the treatment of tinnitus. An unexpected finding has been the response of the first patient in the USA (with category I tinnitus) to be successfully treated for tinnitus with chronic oral memantine 10 mg BID.

Memantine would be administered orally and chronically in patients with various grades of tinnitus (I-IV), as monotherapy or in conjunction with TRT, and in cochlear disorders. Memantine would be administered concomitantly and prophylactically in patients receiving aminoglycoside antibiotics. Memantine, administered chronically in oral doses of 5-100 mg/day, advantageously 10-30 mg/day (serum levels ranging from 0.25-2.0 μg/ml) is efficacious in the treatment of tinnitus, cochlear disorders, and drug-induced ototoxicity. Thus, Memantine administered in conjunction with current standard medical treatments, will be efficacious in preventing and reducing neuronal injury and death in patients with tinnitus, cochlear disorders, and drug-induced ototoxicity. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or neural stem cell implantation.

Nystagmus

Nystagmus is defined as rapid, involuntary ocular movements and consists of multiple types (horizontal, vertical, ocular, rotary). APN (acquired pendular nystagmus) is characterized by oscillopsia and impairment of static vision in clinical conditions such as brain tumors, posterior fossa ischemia, and multiple sclerosis. Evidence that NMDA receptor dysfunction contributes to the etiology of nystagmus has been theorized by both gabapentin and memantine producing efficacy in treating APN in multiple sclerosis patients. Evidence for the role of glutamate in visual oculomotor function is based partly on the oculomotor nucleus (III) where quantitative analysis has revealed a ratio of lower NMDA densities and elevated densities of AMPA receptors. In addition, the NMDA antagonist ketamine has been shown to produce deficits in smooth pursuit of eye movements in healthy subjects, suggesting a role for NMDA receptor in normal gaze functioning. While the efficacy of gabapentin treatment for acquired nystagmus in MS patients has been attributed to an NMDA etiology, those skilled in the art will realize that there is no evidence that gabapentin interacts at the NMDA receptor.

Memantine administered intravenously or orally and chronically in patients with nystagmus, as monotherapy, in conjunction with other medications that either attenuate glutamate or block KA and AMP receptors, would provide both efficacy and safety. Memantine, administered chronically in oral doses of 5-100 mg/day, advantageously 10-30 mg/day (serum levels ranging from 0.25-2.0 μg/ml) is efficacious in the treatment of nystagmus. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or neural stem cell implantation.

Bowel Syndromes

GI diseases include at least inflammatory bowel disease, peptic ulcer, irritable bowel syndrome and functional bowel syndromes. Autonomic control of the GI system includes the SNS (sympthatic), PNS (parasympathetic), and enteric nervous system, the latter consisting of sensory and motor neurons in the GI tract that the mediate digestive reflexes. The SNS consists of the sympathetic preganglion (celiac, superior mesenteric, and inferior mesenteric) neurons along the spinal cord while the PNS, via the vagus nerve, inervates the stomach, pancreas, and small intestine. The enteric nervous system controls the function of the GI, pancreas and gallbladder and is composed of local sensory nerves, interneruons and motor neurons. This system responds to alterations in smooth muscle tension of the gut, chemical environment in the gut, vascular supply and mucosal secretion. During peristalsis, the PNS stimulates the enteric neurons by the nicotinic receptor and contracts smooth muscle by the muscarinic receptor. Nitric oxide is postulated to mediate smooth muscle relaxation in peristalsis.

Inflammatory mediators can sensitize primary afferents (C-fiber nociceptors) and produce secondary spinal sensitization. Chemical mediators such as bradykinin and PGE2 directly activate nerve endings and trigger algesic mediators such as histamine, serotonin, and NGF. Mast cells and platelets play a crucial role in pain transmission with contributions from macrophages and neutrophils. The spinal cord mediates painful GI sensations while substance P, dynorphins, and glutamate function in post-synaptic sensitization, particularly during and after gut inflammation. Thus, alterations in neuroimmune communications at the gut level trigger events that produce changes in visceral and spinal cord sensitivity. Abdominal pain is the most frequent complaint of patients with functional bowel disorders. Mechanisms of hyperalgesia include sensitization of primary afferent nerve endings, enhanced transmission of nociceptive inputs in the spinal cord, alterations in integrative processes of nociceptive messages to the cortex, and defects in the activation of descending anti-nociceptive pathways. Inflammation can produce nerve remodeling and trigger chronic submucosal hypersensitivity by: (a) direct activation of receptors opening Ca++ or Na++ ion channels, (2) up- or down-regulation of receptors in nerve endings associated with changes in numbers and the proximity of resident immunocytes; as well as (3) size and altered distribution of sensory neural endings. Sensitization at the DRG (dorsal root ganglion) level is due to permanent activation from locally released direct and indirect algesic mediators. This hyperexcitability state is important because the ability to amplify nociceptive inputs (or wind-up phenomenon) is persistent and contributes to the pathogenesis of hyperalgesia.

FGD (functional GI disorder) is characterized by abnormal GI responses and enhanced perceptual responses to visceral stimuli from either a central or peripheral etiology. These patients have an increased incidence of anxiety, panic disorder, sleep disorders, and depression. Hypersensitivity of the GI tract is associated with ANS dysfunction, abnormal fluid and electrolyte absorption, and abnormal motility. Genetic factors, psychosocial stressors, and PTSS also influence the clinical expression. Current treatments include GI bulking agents, prokinetics (5HT4 and 5HT3 drugs), smooth muscle relaxants, antispasmodics (anticholinergics and Ca++ channel blockers), tricyclic antidepressants to reduce chronic pain and depression, and anxiolytics. Other potential treatments include selective antagonists of M3 muscarinic receptors, 5HT4 antagonists, peripheral acting kappa opiod agonists, 5HT3 antagonists, neurokinin-1 and CGRP. In recent human clinical trials, the Ca++ channel blockers Diltiazem and Verapamil produced no significant results in efficacy.

Memantine administered intravenously or orally and chronically in patients with BS, as monotherapy, in conjunction with other standard treatments or medications that attenuate glutamate or block KA and AMP receptors, would provide efficacy and safety. Memantine would be administered concomitantly and prophylactically in patients at risk for relapse. Memantine, administered chronically in oral doses of 5-100 mg/day, advantageously 10-30 mg/day (serum levels ranging from 0.25-2.0 μg/ml) is efficacious in the treatment of the clinical and pain symptoms of BS. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or neural stem cell implantation.

Peripheral Neuropathy

Symptoms of PN include motor dysfunction, sensory loss, decreased reflexes, and autonomic dysfunction. Presentation may be acute or subacute, chronic, relapsing, symmetrical or asymmetrical. Clinical subtypes include polyneuropathy, polyradiculopathy, neuronopathy (motor or sensory), multiple mononeuropathies, or plexopathies. Neuropathies can be classified as: (1) idiopathic inflammatory (GBS, CIDP); (2) metabolic (diabetic, renal) or nutritional (B₁₂); (3) infectious (diptheria) or granulomatous (sarcoid); (4) vasculitic (PAN, RA, SLE); (5) neoplastic or paraproteinemia; (6) drugs (dilantin) or toxins (chemotherapy, metals, organic phosphates); and (7) hereditary neuropathies. Basic pathological processes that destroy the peripheral nerve include wallerian degeneration (degeneration of the axis cylinder and myelin distal to the axonal injury), segmental demyelination (axon sparing), and axonal degeneration (distal degeneration of both myelin and axis cylinder). Axonal transport is bi-directional and occurs in both anterograde and retrograde, and is classified as either fast or slow transport. In peripheral neuropathy (type), both abnormal axonal transport and abnormal glutamate metabolism have been demonstrated, and these deficits were shown to be reversed by NMDA antagonists. Peripheral neuropathy eventually produces significant disability including gait disturbances, Charcot's joints, and autonomic dysfunction.

A prior patent claim (Lipton U.S. Pat. No. 5,334,618) of memantine is treating painful peripheral neuropathy due to a central etiology. Those skilled in the art will recognize that this claim is a treatment for chronic pain whose etiology is assumed to be in the spinal cord or brain sensory area in a patient with early painful PN. Those skilled in the art will recognize that while pain may be a component of peripheral neuropathy, it is not a universal phenomena of peripheral neuropathy and that pain may abate with severe degeneration of peripheral nerves. In addition, those skilled in the art will recognize that the pain from peripheral neuropathy is not a primary CNS disorder but that the chronic pain from peripheral neuropathy may cause a secondary abnormal neuronal plasticity in the spinal cord (wind up) and increased sensitivity of the limbic system and cortex. We hypothesize a new NMDA dependent mechanism of peripheral neuropathy that originates in various subtypes of distal peripheral nerves, a mechanism where NMDA dysfunction in the peripheral nerves alters spinal cord and brain NMDA and non-NMDA function, and also hypothesize a glutamate receptor dependent peripheral mechanism of pain generation.

An increase in NMDA receptor density has been reported in injured neurons after peripheral axotomy. Injured neurons have been shown to have an increased susceptibility to NMDA-induced neurotoxicity while MK-801 reduced motorneurone death following nerve injury. These results suggested that neuronal vulnerability to excitotoxic damage occurred by mechanisms that caused increased neuronal Ca++ influx that can occur at lower neurotransmitter concentrations. We hypothesize that abnormal NMDA density or altered NMDA receptor subtype composition also increases neuronal vulnerability to excitotoxic stress.

For example, altered binding density of AMPA receptors in the upper thoracic spinal cords of obese rats, impaired modulation of the AMPA receptor in streptozotocin-treated rats in diabetic neuropathy, and decrements in brain AMPA receptor density have been reported. In these animals, inhibition of tactile allodynia was produced by both NMDA and AMPA receptor antagonists suggesting an interaction between these receptors and a role for glutamate in producing these abnormalities. Recent studies have revealed the NMDA R1 subunit on the trigeminal and dorsal root ganglion while both unmyelinated and myelinated axons in the sural nerve (sensory) and medial plantar (sensory and motor) nerve also contain NMDA, AMPA and KA receptor units. These findings are consistent with the observation that both NMDA and non-NMDA antagonists have been shown to ameliorate nociceptive behaviors from noxious peripheral stimulation. In the sural nerve, 48% of the myelinated axons and 21% of the unmyelinated axons contained the NMDA R1 subunit while in the medial plantar nerve, 56% of the myelinated fibers and 30% of the unmyelinated nerves contained the NMDA R1 subunit. The presence of glutamate receptors on large-diameter myelinated axons, Aδ and Aβ, suggest that these mechanoreceptors (transducing touch and pressure) are chemosensitive and respond to local glutamate. We hypothesize that elevated serum and tissue glutamate and inflammatory mediators produce an excessive stimulation of the peripheral nerve NMDA and non-NMDA receptors resulting in neuropathy. Up-regulation and excessive activation of glutamate receptors in the spinal cord may be secondary to sensory and motor neuropathy, such as in diabetes. Using quantitative auto-radiography for NMDA and AMPA receptors in the thoracic spinal cord in lean and obese-diabetic mice, increased binding sites and affinity for the NMDA receptor was found to be significantly higher in obese mice. Thus, increased expression of the glutamate receptor subtypes, and altered ligand affinity for the NMDA receptor subtype in the obese mice reflects the pro-inflammatory state that obesity produces and secondarily contributes to diabetic peripheral neuropathies. In addition, patients with sepsis or who are confined to the ICU for prolonged periods of time also develop a generalized peripheral neuropathy. While the etiology is currently unknown, we hypothesize a systemic and local inflammatory upregulation that stimulated peripheral glutamate receptors.

Memantine will be administered orally and chronically in patients with moderate or severe peripheral neuropathy, at risk for peripheral neuropathy but asymptomatic, or with early mild symptoms. Memantine as monotherapy, in conjunction with other standard treatments, or medications that attenuate glutamate or block KA and AMP receptors, would provide efficacy and safety. Memantine, administered chronically in oral doses of 5-100 mg/day, advantageously 10-30 mg/day (serum levels ranging from 0.25-2.0 μg/ml) is efficacious in the treatment of the clinical symptoms (excluding the indication of pain) and morbidity, as well as the attenuation of the progression of peripheral neuropathy from the various etiologies of peripheral neuropathy. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or neural stem cell implantation.

Metabolic Bone Diseases and Osteoporosis

Bone mass is regulated by multiple factors including mechanical factors, osteotropic hormones (calcitonin and parathyroid hormone), cytokines, nitric oxide, and glutamate receptors. Glutamate transporters (GLAST and GLT-1) have been located in bone, suggesting a role for glutamate in paracrine intercellular signaling in bone. GLAST protein is mechanically regulated in both osteocytes and osteoblasts while GLT-1 was localized to the pericellular region of mononuclear bone marrow cells. GLAST is decreased with mechanical loading and increased with activity in both bone and periosteal surfaces suggesting that activity regulates the expression of GLAST.

The expression of NMDA R-1 and 2D subunits as well as PSD-95, a clustering protein associated with NMDA signaling in the CNS, has been identified in bone. NMDA R1 expression has been localized to osteoblasts and osteoclasts while the NMDA R12D subunit was found to be less sensitive to Mg++ block. Glutamate binding to osteoblasts stimulates an increase in intracellular Ca++ while glutamate receptor antagonists inhibit osteoclast differentiation. Excitatory amino acids are known to be chemotactic for marrow-derived cells, including osteoclast progenitors. These findings suggest a physiological role for glutamate in bone.

Glutamate has been shown to bind to osteoblasts while NMDA antagonists (D-APV) inhibits this binding. Both Mg++ and MK-801 caused a significant decrease in inward currents in response to NMDA agonist stimulation. Bone cell signaling by glutamate and paracrine communication between bone cells by NMDA receptor activation is important in bone remodeling. In transgenic mice with decreased expression of the AMPA receptor (GluR2), marked skeletal developmental abnormalities (kyphosis and reduction in skull and long bone growth) occurred. Finally, the enzyme tyrosine kinase c-src is known to interact with the NMDA receptor, and c-src deficient mice have a deficit in osteoclast function. These observations suggest that glutamate receptor mutations have a direct effect on bone formation and implicate a functional role for glutamate in bone physiology.

Using electron microscopy analysis, substantial nerve density has been shown to accompany vessels adjacent to bone trabeculae, in the vicinity of both hemapoetic cells and bone cells. Glutamate expression occurred in a portion of these nerve processes in close proximity to bone cells suggesting a glutaminergic innervation of the bone. Bones also have sympathetic innervation and a high degree of peptidergic innervation in regions of high osteogenic activity. In conclusion, neuronal glutamate transporters and functional subtypes of NMDA receptors in both osteoclasts and osteoblasts suggest a functional regulation glutaminergic innervation of bone with local regulation of bone cell function.

Osteoclasts, the only cells known with the capacity to dissolve crystallized hydroxyapatite and degrade the organic matrix of bone, have a short half-life (t_{1/2}) and undergo apoptosis within days. The short t_{1/2} of osteoclasts are partially due to the low expression of Bcl-2, which blocks apoptosis, while over expression of Bcl-2 has been shown to block apoptosis. Caspases are involved in the regulation of survival and apoptotic cell death of osteoclasts while IL-1α and M-CSF promote osteoclast survival by suppressing the action of caspases. Both estrogen and biphosphonates inhibit bone resorption by promoting and inducing osteoclast apoptosis, and are therefore clinically effective in treating diseases of increased bone turnover. Thus, if bones are not active and signaled by other cells or survival factors by paracrine signaling, an intrinsic death program is activated.

The predominant isoform of nitric oxide (NO) expressed in normal adult bone is the constitutive isoform, eNOS, mainly in osteoblastic cells. NO modulates osteoclast recruitment and activity while osteoblastic cells respond to mechanical strain and shear stress by a rapid increase in nitric oxide production. In an experimental model of inflammatory bowel disease, cancellous bone formation is markedly suppressed in the presence of active colonic inflammation. The induction of iNOS in osteoblasts by cytokines (IL-1, TNF-α, INF) may be the etiology for the suppression of bone formation. Cytokine-induced NO production has been shown to inhibit osteoblast growth and to stimulate osteoblast apoptosis. IL-3 and IL-4 causes inhibition of cell proliferation and enhancement of alkaline phosphatase activity in human osteoblasts. The bone remodeling process is modulated by proinflammatory cytokines, including IL-1, IL-6 and TNF (α and β). IL-I is produced exclusively by activated memory T cells and stimulates osteoclastic resorption by stimulating nitric oxide via the NF-KB nuclear factor. An excess of these cytokines has been postulated to contribute to the development of post-menopausal osteoporosis, bone loss in inflammatory disease, and tumor-induced osteolysis. While the anti-inflammatory cytokines IL-13 and IL-4 down regulate the formation of various proinflammatory cytokines in activated monocytes, mice that over express IL-4 have been shown to develop severe osteoporosis. IL-1p, TNF-α, and IL-6 are bone-resorbing cytokines which increase osteoclastogenesis, the hallmark of postmenopausal and glucocorticoid-induced osteoporosis. In a model of fracture healing, the expression of neurotrophins and trkB receptors in bone forming cells suggests a role in both differentiation and survival of bone-associated neurons and bone formation by autocrine and paracrine mechanisms. The demonstration of neuropeptides in periosteum nerve fibers and neuropeptide receptors on bone cells further suggests that various aspects of bone metabolism are under neural control.

In summary, regulated intercellular signaling is essential for the maintenance of bone mass. Both osteoblasts and osteoclasts express functional glutamate receptors as well as the synaptic specific protein complexes required for regulated glutamate exocytosis in presynaptic neurons. Osteoblast cells actively release glutamate in a differentiation-dependent manner by a presynaptic vesicular exocytosis and mechanisms exist for communication between osteocytes, osteoblasts, and osteoclasts. Since GLAST is also expressed in both osteoblasts and osteoclasts, regulated presynaptic vesicular exocytosis implies a highly targeted glutamate-mediated intracellular signaling between bone cells. Bone is continuously remodeled and bone cell activity is under the influence of systemic factors as well as local growth factors, neuropeptides and cytokines. The identification of glutamate and aspartate receptors in bone further suggests a role of neuroexcitatory amino acids in bone cell paracrine signaling. NMDA antagonists may have efficacy in osteoporosis, fracture healing, osteoporosis from prolonged weightlessness or bed rest, metastatic disease, spinal cord disease and injury, chronic steroids use, etc. Since memantine is an NMDA receptor antagonist with preferential activity on NR2C and NR2D subunits, memantine has significant efficacy in treating various bone disorders.

Memantine, administered orally, chronically and prophylactically in patients with or at high risk for bone disorders (i.e., chronic seizure treatment) in oral doses of 5-100 mg/day, advantageously 10-30 mg/day (serum levels ranging from 0.25-2.0 μg/ml) is efficacious in decreasing morbidity and mortality of osteoporosis and other metabolic bone disorders. Memantine, when administered in conjunction with current standard medical treatments, will be efficacious in preventing and reducing clinical manifestations of osteoporosis and other metabolic bone disorders. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or stem cell implantation.

Pulmonary Disorders

NMDA receptors have been located in the respiratory system distal to the larynx. In addition, about 90% of tachykinin-containing sensory neurons are known to contain glutamate. NMDA receptor activation in perfused, ventilated rat lungs produces an acute injury characterized by increased ventilation-perfusion pressures and high-permeability edema. The onset of pulmonary edema was correlated with an increase in airway resistance. These findings suggest that excessive activation of NMDA receptors may induce acute edematous lung injury or ARDS (adult respiratory distress syndrome). This lung injury was prevented by competitive NMDA antagonists, channel-blockers (MK801), reduced in the presence of Mg++, and was nitric oxide (NO) dependent since it was attenuated by NO synthase inhibitors. Pulmonary injury was also decreased by VIP (vasoactive intestinal polypeptide) and inhibitors in PARP (poly ADP-ribose polymerase) that inhibit NO toxicity. The observation that VIP protects against pulmonary glutamate toxicity may be attributable to anti-oxidant activity, inhibition of PARP activation, and upregulation of bcl-2 expression.

In an animal model, intrathecal injection of capsaicin was shown to reproduce various cardinal features of bronchial asthma. Capsaicin is known to act on sensory afferent C-fibers to release proinflammatory tachykinins that produce smooth muscle constriction, increase vascular permeability, and induce plasma exudation. The acute elevation in airway perfusion pressure was attenuated in both magnitude and duration by MK-801. NMDA receptor activation increases resting muscle tone and enhances the contractile response to acetylcholine while this increased airway perfusion pressure produced by NMDA was abolished by MK-801. We hypothesize that NMDA receptor activation is an important mechanism of both airway inflammation and hyper-activity found in bronchial asthma and other pulmonary diseases. These mechanisms explain the clinical observation of the triggering and exacerbation of acute asthma attacks by glutamate-containing foods and the relaxant effect of ketamine on airway smooth muscle. Finally, glutamate may produce pulmonary cell death by both apoptosis and necrosis in the lung.

In acute human domoic acid toxicity, 12 of 19 hospitalized patients required ICU (intensive care) admission for symptoms of coma, seizures, unstable blood pressure, and profuse pulmonary secretions. Of these, 9 patients required intubation to protect their airways from profuse secretions, but not from respiratory failure. These findings suggest a role for glutamate in the clinical expression and severity of pulmonary diseases. We further hypothesize that the clinical expression of neurogenic pulmonary edema (NPE) has contributions from central NMDA receptor activation in the respiratory center of the brain. Thus, both central and peripheral NMDA receptor activation may contribute to the clinical expression of various pulmonary diseases.

We hypothesize that the modulatory role of memantine on NMDA receptors will attenuate the clinical symptoms in pulmonary conditions such as pulmonary edema, neurogenic pulmonary edema, bronchial asthma, ARDS, and other respiratory diseases. In addition, the degree of apoptosis and necrosis will also be attenuated. Memantine, administered acutely by the intravenous route or chronically in oral doses of 5-100 mg/day, advantageously 10-30 mg/day (serum levels ranging from 0.25-2.0 μg/ml) is efficacious in the treatment and prophylaxis of these pulmonary disorders. Memantine may be administered concomitantly with current standard medical treatments for pulmonary disease. The duration of memantine treatment well be determined by clinical parameters but may be chronic and indefinitely. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or stem cell implantation.

Obesity and Complications of Obesity

Obesity is a chronic condition characterized by an excessive accumulation of adipose tissue, which can occur through an increase in adipose cell size, cell number, or both. Excess energy is usually stored as triglycerides in adipose cells. While obesity is due to a combination of increased caloric intake and sedentary lifestyle, genetic factors may be responsible for the variation in 40-70% of obesity phenotypes. Obesity is an increasingly common disorder which may affect up to 50% of the population, of which 20-25% are severely obese and 10-15% are morbidly obese. Obesity is usually calculated by the BMI (body mass index or weight in kilograms divided by height in meters squared) with a value>30 considered obese and values>35-40 considered morbidly obese. In a female study, there was a 100% greater risk of death from all causes for a BMI>30 kg/m² compared to a BMI<19 kg/m². Besides mortality from obesity, other health complications include insulin resistance, diabetes, hypertension, sleep apnea, hyperlipidemia, cerebral hemorrhage, pseudotumor cerebri, cancer, osteoarthritic spine and joint disease, cholecystitis, and coronary artery and cardiac disease.

The exact etiology of obesity in unknown but we hypothesize that neurological mechanisms play a predominant role. Appetite control and satiation requires complex interactions but involves multiple neurotransmitters and neuropeptides in the hypothalamic nuclei and limbic system as well as frontal lobe inhibition. Genetic predisposition contributes to the amount and distribution of body fat. In addition, genetic obesity disorders include such diseases as Prader-Willi, Laurence-Moon-Biedl, Alstrom, Cohen, Carpenter and Blount's syndrome.

Recent neurochemical brain research has implicated multiple neurotransmitters that stimulate (neuropeptide-Y or NPY, GABA, galanin, noradrenaline etc.) and inhibit appetite (leptin, GLP-1, CRF, neurotensin, melanocortin). The levels of circulating leptin has been suggested to correlate with fat mass, while inhibition of NPY secretion (the most potent appetite stimulant) appears the mechanism by which leptin decreases food intake. NPY has multiple receptor subtypes (Y1-Y6) with Y1 and Y5 involved in feeding behavior. NPY has also been shown to selectively suppresses excitatory transmission by inhibition of presynaptic glutamate release mediated by Y2 receptors. Thus, we hypothesize that appetite regulation and obesity may be due to hypothalamic glutamate dysregulation.

In a study of morbidly obese humans, plasma leptin levels concentrations correlated with increased levels of inflammatory indices. Thus, BMI correlated with leptin, acute phase proteins, TNF-α receptors, and plasminogen activator inhibitor-1 (PAI-1) suggesting that the condition of obesity produces a pro-inflammatory state. Importantly, leptin and TNF-α concentrations were strongly correlated, indicating that leptin has a regulatory role in the degree of inflammation in obese patients. Since glutamate receptors regulate the release of insulin from the pancreas and TNF-α is toxic to the islet cells, we further hypothesize that the induction of a pro-inflammatory state by obesity contributes to the conversion of type II diabetes to insulin-dependent diabetes.

In animal studies, blocking the NMDA receptor produces both the suppression of appetite and a decrease in weight (even in the presence of starvation), suggesting that glutamate may also be involved in the pathophysiology of obesity. In human studies where rCBF was measured by PET scanning, satiation in obese females produced greater increases in the ventral prefontal cortex and significantly greater decreases in the paralimbic areas, frontal and temporal cortex. Importantly, rCBF was significant in the hypothalamus, cingulate, nucleus accumbens, and amygdala only in obese females. Abnormal rCBF has also been reported in females with binge-eating disorders as well as bulimic females, maximally in the frontal, prefontal and temporal brain regions. Since lesions of the amygdala can result in hyperphagia and obesity, we postulate that abnormal quantities or function of non-NMDA and NMDA receptors in the prefontal cortex, amygdala and hypothalamus are involved in the clinical expression of chronic obesity. We further hypothesize that NMDA regulation in the these brain areas by memantine may modulate neuropeptide-Y and other factors that would subsequently result in weight loss and attenuation of the pro-inflammatory state of obesity. Additional evidence of altered glutamate and glutamate receptor function is the finding of abnormal NMDA receptor expression in the spinal cord in obese mice.

Memantine would be administered orally and chronically in patients diagnosed with mild, moderate or morbid obesity. Memantine, administered chronically in oral doses of 5-100 mg/day, advantageously 10-30 mg/day (serum levels ranging from 0.25-2.0 μg/ml) is efficacious in controlling obesity, preventing the development of insulin-dependent diabetes, and the medical complications of chronic obesity. Memantine, will be administered in conjunction with current standard medical treatments, will be efficacious for the treatment of the acute and chronic neurological complications of obesity. Memantine may also be used in combination with glycine-site NMDA antagonists, AMPA antagonists, calcium channel blockers, anti-oxidants, anti-inflammatory drugs, caspase inhibitors, calpain inhibitors, neurotrophins (NT3, BDNF), or stem cell implantation.

Diabetes and Pre-Diabetes

DM-I results from selective destruction of the insulin-producing β cells in the pancreatic islets of Langerhans. A current theory of DM-I is that β cells are destroyed by an autoimmune response mediated by T lymphocytes (T cells) that react specifically to one or more B cell proteins (autoantigens). Support for this concept includes: a slower progression of islet β-cell damage in recent onset DM-I with immunosuppressive agents, the islet lesion (insulitis) infiltrated by lymphocytes, macrophages or monocytes, and the co-existence of DM-I with other autoimmune diseases, notably thyroiditis. DM-I is associated with alleles of the HLA gene that regulate immune responses. In DM-I, the immune system inappropriately attacks healthy β-cells or a primary β-cell lesion (viral or chemical) initiates an autoimmune response. Thus, DM-I develops from a disorder of immunoregulation, allowing β-cell autoreactive T cells to become activated, expand clonally, and induce a cascade of immune and inflammatory processes (insulitis), culminating in β-cell destruction.

Diabetes mellitus is a serious endocrine disorder with disruption of intermediary metabolism due to insufficient insulin secretion, activity, or both. The term includes DM (type 1 and type 2), impaired glucose tolerance (GTT), syndrome X, pre-diabetes, and diabetes secondary to pancreatic disease, hormonal alterations, or genetic syndromes. DM-I usually occurs before the age of 35 years. An abrupt onset of symptoms, a high frequency of ketoacidosis (KA) and the presence of autoantibodies directed against insulin distinguish DM-I in early childhood. Older children with DM-I may exhibit high levels of autoantibodies directed against pancreatic β-islet cells or glutamic acid decarboxylase (GAD), while adult DM-I have lower levels of these autoantibodies.

Antigen-activated T cells are termed T helper (Th) cells because they mediate both cellular and humoral (antibody) immune responses. Each has a distinct cytokine secretion pattern: Th1 secretes IL-2, IFN-γ, and TNF-β while Th2 secretes IL-4, IL-5, and IL-10. Thus, a combination of genetic and environmental factors produce a disease susceptibility that creates a pathogenic immune response wherein autoreactive T cells produce insulitis. Macrophages producing IL-1 and T cells producing TNF α/β and INFγ are postulated to produce toxic oxygen and nitrogen free radicals. Th1 cell formation is pathogenic for DM-I with the secretion of IL-2 and IFNγ producing macrophages resulting in β-cell attack and death. However, while the proinflammatory cytokine IFN-α has been detected in β-cells of patients with recent onset of DM-I, the stimulus is unknown.

Familial aggregation of DM-I occurs with a relatively low rate of concordance between MZ twin pairs (50%). The HLA region on chromosome 6, which encodes for gene products associated with immune response regulation, accounts for 40% of the familial inheritance. HLA-DR3 and -DR4 alleles strongly associated with DM-I while the DR2 and DR 5 alleles have protective effects. DM-I also has an association (10%) with chromosome 11 as well as the CTLA-4 gene on chromosome 2, which encodes a receptor that mediates T-cell activation that is implicated in the autoimmune pathogenesis of DM-I.

Environmental trigger factors are postulated since 90% of DM-I occurs in the absence of any family history. The onset of β-cell damage may occur years prior to the emergence of overt symptoms and environmental triggering factors may be present during gestation or after birth. Viral infections are implicated since elevated levels of antibodies to the Coxsackie B enterovirus have been documented at childbirth in mothers of children who became diabetic prior to the age of 15 years. Coxsackie B antibodies are more prevalent in newly diagnosed DM-I patients aged 3-14 years than in controls while Coxsackie B virus RNA sequences have been detected in 42% of newly diagnosed adult DM-I patients. Thus, insulitis and β-cell destruction in vivo by Coxsackie virus may be due to the cross reactivity (molecular mimicry) between homologous sequences in the virus and the β-cell autoantigen, GAD. A higher frequency of DM-I also occurs in young adults with congenital rubella syndrome, an autoimmune virus that increases the concentration of islet cell antibodies and enhances the inflammatory cascade in β-cells by elevating cytokines such as IL-1 and IL-6. Conversely, recent viral model evidence suggests that neither B lymphocytes nor antibodies to islet cells had a direct role in the pathogenesis of DM.

DM-II is the predominant late-onset form of the disease (90%) and a subtype called maturity onset diabetes of the young (MODY) is characterized by impaired insulin secretion without KA. DM-II is characterized by relative insulin deficiency due to abnormal insulin secretion and insulin resistance in target tissues. Pancreatic β-cells remain anatomically intact, although they are unable to compensate for the body's reduction in sensitivity to insulin. A concordance rate for DM-II in monozygotic twins of 70-80% and a risk of DM-II in the offspring of two parents with the disease of 70% demonstrate genetic factors, but common DM-II has a strong complex polygenic mode of inheritance. MODY is an autosomal dominant form (of which there are four subtypes) of early onset DM-II characterized by a primary defect in insulin secretion. Finally, the Pima Indians of Arizona, a genetically homogenous group, have the highest reported prevalence of DM-II with over half the population developing the disease after the age of 35 years.

Other risk factors for DM-II include insulin resistance (IR), obesity, sedentary life style, low birth weight, and aging. IR is a consistent risk factor for the development of DM-II and the etiology of the controversial “Syndrome X”, a condition manifested by hypertension, dyslipidemia, and metabolic abnormalities that increase the risk of cardiovascular disease. IR is an early development in disease pathogenesis, as reductions in insulin sensitivity may occur for a decade prior to the emergence of overt disease and has been postulated as a primary defect, leading to β-cell exhaustion and a deficiency in insulin secretion. Severe IR due to mutations in the insulin receptor gene can result in DM-II (despite normal levels of insulin secretion) while the degree of IR predicts the progression of glucose intolerance to DM-II. Elevated levels of plasma free fatty acids and abdominal or central adiposity increases the risk of DM-II in obesity by causing IR. Age increases the prevalence of DM-1 μl from 10% over the age of 60 to 16-20% over the age of 80 years due to elevations in fasting plasma insulin (IR), alterations in β-cell number and function, and reductions in glucose tolerance.

Complications of DM include both micro- and macroangiopathy. Microangiopathy is intimal thickening in small arterioles, leading to disruption of local autoregulation and producing retinopathy, nephropathy and neuropathy. Mechanisms include increased glycation of proteins, increased polyol metabolism via aldose reductase, and the generation of oxidative stress. Glucose-induced activation of protein kinase C (PKC) is also implicated in increased vascular permeability, cell proliferation, and abnormal retinal and renal hemodynamics. Macroangiopathy produces atherosclerosis in coronary, peripheral, and cerebral arteries that are clinically manifested by ischemic heart disease, peripheral vascular disease, and stroke. Diabetes induces earlier and progressive atherosclerosis and increases the risk of cardiovascular disease by two to five times. AGEs (advanced glycation end products) contribute to the pathogenesis of macroangiopathy while elevated levels of glycated lipoproteins (LDL) are engulfed by macrophages, producing foam cells, an early characteristic of atherosclerosis. Moreover, glycated LDL is more susceptible to toxic oxidative processes and increases platelet aggregation, both of which are atherogenic and contribute to macroangiopathy and atherosclerotic fibrous plaques. A lipid profile of increased triglycerides, smaller LDL particle size, and decreased levels of HDL are observed in non-diabetic patients with insulin resistance, suggesting a relation between IR and dyslipidemia. Finally, reduced receptor-mediated clearance of LDL has been linked to the formation of AGEs in vivo.

Both DM-I and DM-II are associated with cerebral dysfunction manifested primarily by mild cognitive impairment in the absence of ischemia or hypoglycemic reactions, with the duration of illness an important factor. Post-mortem analyses have suggested that chronic and poorly controlled diabetes is associated with degenerative changes in the brain. A current theory is that poor glycemic control potentiates pathological cell death (amyloid deposition) and accelerated aging or apoptosis. Brain MRI in asymptomatic DM-II patients with multiple risk factors (age, HTN and hyperlipidemia) revealed lacunae in 42% of DM-II patients. Global measures of cognitive dysfunction correlated with the presence of lacunae. PET studies of ¹⁸FDG cerebral glucose consumption revealed significant reductions in chronic diabetes (with symptoms of peripheral neuropathy) when compared to newly diagnosed patients. Importantly, cerebral glucose metabolism was inversely correlated with both the duration of diabetes and age. Severe hypoglycemic episodes and hypoglycemic unawareness have also been reported to produce permanent neuropsychological impairment in DM, while depression is greater in DM-II patients. An analysis of published studies on cognitive dysfunction and DM-I concluded that most studies reported that diabetic patients had deficits in measures of higher cognitive abilities compared to control subjects. These observations are corroborated by an analysis of CBF in situations requiring increased brain metabolic demand, where normal increases occurred in controls (86%) but not diabetic patients (39%).

The onset of the clinical symptoms of DM is preceded by a preclinical stage lasting months to years, during which evidence of both pancreatic islet cell autoimmunity and defective insulin secretion may be detected. Studies in identical twins and asymptomatic first-degree relatives of DM-I patients reveal the presence of ICA (insulin cell antibodies), IAA (insulin autoantibodies), tyrosine phosphatase (IA 2Ab) and GAD autoantibodies all of which increase the risk of DM-I. Specifically, the combination of GAD and IA2 in first-degree relatives is highly predictive of clinical diabetes, while higher PAI (plasminogen-activator-inhibitor) levels also predict the development of DM. The first phase insulin release (FPIR) to an intravenous glucose challenge is also highly predictive of DM in antibody positive relatives. In a study, the risk of DM within five years is 85% in ICA positive relatives with an FPIR of <50 mU/L. These results suggest that DM-I can currently be diagnosed in the preclinical stage with the best markers being GAD, IA2, and FPIR as screening tools in at risk patients and first-degree relatives.

There is currently a focus on identifying candidates for intervention treatment in the preclinical stage, where loss of β-cell function is less advanced. The ability to identify individuals in the pre-diabetic stage provides an opportunity to prevent the autoimmune destruction of pancreatic cells. Strategies under consideration for the prevention of DM-I include the induction of immunotolerance and the prophylactic protection of β-cells. Therapies include the suppression of immune responses is thought to be the shifting of autoimmune T-helper cell (Th) from a destructive (Th1) to a protective (Th2) profile. Various cytokines or cytokine inhibitors may direct the immune response from self-aggression to self-tolerance. As pancreatic β-cell destruction is mediated by the generation of cytokines and free radicals antioxidant therapy may prevent or limit cell death. Amylin, an amino acid peptide hormone secreted with insulin from pancreatic β-cells, functions to reduce postprandial glucose levels by suppression of glucagon secretion and modulation of gastric emptying. Since amylin levels are reduced or absent in patients with DM-I or late DM-II, replacement therapy (i.e., pramlintide) has emerged as a potential option to improve glycemic control. GLP-1 is released into the bloodstream following meals and suppresses postprandial hyperglycemia. The truncated form of the gut hormone, GLP-1, has demonstrated multiple anti-hyperglycemic effects in diabetic patients, including the enhancement of insulin secretion in response to glucose, slowing of gastric emptying, and suppression of glucagons production. Pimagedine (aminoguanidine) inhibits the formation of AGEs in diabetes and also inhibits NOS (nitric oxide synthase) and oxidative stress. Bimoclomol, a hydroxylamine derivative, is a novel cytoprotective agent that induces the in vivo expression of heat shock proteins (HSP). These HSPs maintain cell integrity under pathophysiological glycemic conditions and thus may prevent complications of diabetes. NAD (Nicotinamide) is a soluble B-group vitamin that has been shown to improve β-cell regeneration in models of DM (spontaneous and induced), increase insulin synthesis, and prevent development of clinical DM in animal models if administered before onset. Postulated mechanisms include: (1) inhibiting poly-ADP-ribose polymerase (a major route of NAD metabolism), (2) serving as a free radical scavenger, and (3) inhibiting cytokine-induced islet nitric oxide production. Additional therapies include cytokines, antibodies to cytokines or cytokine receptors, soluble cytokine receptors, and receptor antagonists.

It has been shown that AMPA and NMDA receptors are required for the release of insulin. In addition, recent evidence suggests that obesity produces a pro-inflammatory state and this may be a function of leptin, whose concentration is a function of degree of obesity. We hypothesize that a major component of the development is an inflammatory action that destroys the NMDA and AMPA receptors in the islets, resulting in DM. Thus, administering memantine to block the NMDA receptor in patients at risk, first-degree relatives or newly diagnosed patients with some residual islet cell function will prevent the development of the DM syndrome. Memantine will be administered with insulin (continuous infusion pumps, oral, intrapulmonary, intranasal, transferal, buccal, β-cell implantation) or oral hypoglycemic agents, or with other NMDA or AMPA receptor antagonists, or with novel therapies such as immunotolerance, cytokines and cytokine receptor antagonists, antioxidants, amylin, GLP-1, pimagedine, bimoclomal, and NAD.

Memantine would be administered orally and chronically in patients at risk, first-degree relatives or newly diagnosed DM. Memantine, administered chronically in oral doses of 5-100 mg/day, advantageously 10-30 mg/day (serum levels ranging from 0.25-2.0 μg/ml) is efficacious in controlling DM-I and DM-II, preventing the development of insulin-dependent diabetes, and the medical complications of chronic diabetes. Memantine, will be administered in conjunction with current standard medical treatments (insulin and oral hypoglycemic agents), will be efficacious for the treatment of the acute and chronic neurological complications of DM. Memantine may also be used in combination with glycine-site NMDA antagonists, AMPA antagonists, calcium channel blockers, anti-oxidants, calpain inhibitors, anti-inflammatory drugs, neurotrophins (NT3, BDNF), or stem cell implantation.

What is claimed:

1. A pharmaceutical compostion which comprises a pharmaceutically effective amount of a compound selected from the group consisting of memantine, felbamate, acamprosate, MRZ 2/579, and mixtures thereof.

2. A method of treatment or prophylaxis of a neurological disease, condition or syndrome comprising administering to a patient a pharmaceutically effective amount of a compound selected from the group consisting of memantine, felbamate, acamprosate, MRZ 2/579, and mixtures thereof.

3. The method of claim 2 wherein said neurological disease, condition or syndrome is selected from the group consisting of neuroprotection in epilepsy, familial Alzheimer's disease (FAD), minimal cognitive impairment (MCI), Down's syndrome, normal cognitive senescence, meningitis, sepsis and septic encephalopathy, CNS vasculitis, schizophrenia, drug and opiate addiction, alcoholic diseases, multiple sclerosis, leukodystrophies and X-ADL, childbirth and surgical anesthesia, traumatic brain injury, spinal cord injury, hypoglycemia, encephalopathy, tumors and malignancies (brain, spinal cord, and systemic), cerebellar degenerations, and ataxias, pre-clinical Huntington's disease, depression, neuroprotection from cerebrovascular risk factors and post-ischemic neurovascular syndromes, migraine, vertigo, tinnitus and cochlear disorders, bowel syndromes, peripheral neuropathy, metabolic bone disease and osteoporosis, obesity, and diabetes and pre-diabetic syndromes.

4. Compositions and methods for the prevention and/or decrease in progression of acute or chronic neurological disorders that involve excessive activation of the NMDA receptor, which compositions are relatively non-toxic, have high degree of effectiveness and continue to produce a therapeutic response over a prolonged period of time.

5. Compositions and methods for the treatment of acute and chronic neurological disorders in humans that involve excessive activation, increased or decreased NMDA receptor density, abnormal NMDA subunit composition, abnormal NMDA receptor binding kinetics, or hypofunction of the NMDA receptor.

6. Compositions and methods effective to control or attenuate acute or chronic neurological disorders utilizing compounds that act as non-competitive antagonists of the open channel of the NMDA receptor, either at the Mg++ site or at an independent site.

7. Compositions and methods effective to prophylactically treat or prevent progression of acute or chronic neurological disorders.

8. A method for the attenuation of neuronal death in diseases or neurological diseases (presymptomatic) that cause loss of cognitive function, by preventing neuronal death by excessive activation or hypofunction of the NMDA receptor.

9. A method for the attenuation of apoptosis or necrosis in diseases or neurological diseases (presymptomatic, acute, subacute or chronic) that cause loss of neuronal death, by preventing neuronal death by excessive activation or hypofunction of the NMDA receptor.

10. A method for the treatment of diseases or neurological diseases (presymptomatic, acute, subacute or chronic) by normalizing NMDA receptor function in conjunction with other standard medical treatments for that particular disease.

11. A method for the treatment of diseases or neurological diseases (presymptomatic, acute, subacute or chronic) that combine preventing hypofunction (under stimulation) or excessive NMDA receptor activation at the open-channel with other forms of neuroprotection: glycine-site NMDA inhibitors, inhibitors of glutamate release or synthesis, AMPA and kainate inhibitors, polyamine inhibitors, inhibitors of NO (nitric oxide) synthesis, GABA inhibitors, anti-oxidants, acetylcholinesterases, nootropic drugs, calpain inhibitors, or the addition of various nerve growth factors.

12. Methods for the attenuation and treatment of diseases or acute and chronic neurological disorders by providing intravenous, transdermal, rectal, oral routes (including sustained or extended release formations) or modified drug delivery systems (such as lipid emulsion or crystal technology) of administration that prevent excessive activation or hypofunction of the NMDA receptor by acting at the open-channel.

13. A method of treatment where the compounds consist of memantine, felbamate, acamprosate, and MRZ 2/579. The compounds will be used as monotherapy or in various combinations with each other.

14. A method of treatment or prophylaxis for the following diseases or syndromes: neuroprotection in epilepsy, familial Alzheimer's disease (FAD), minimal cognitive impairment (MCI), Down's syndrome, normal cognitive senescence, meningitis, sepsis and septic encephalopathy, CNS vasculitis, schizophrenia, drug and opiate addiction, alcoholic diseases, multiple sclerosis, leukodystrophies and X-ADL, childbirth and surgical anesthesia, traumatic brain injury, spinal cord injury, hypoglycemia, encephalopathy, tumors and malignancies (brain, spinal cord, and systemic), cerebellar degenerations, and ataxias, pre-clinical Huntington's disease, depression, neuroprotection from cerebrovascular risk factors and post-ischemic neurovascular syndromes, migraine, vertigo, tinnitus and cochlear disorders, bowel syndromes, peripheral neuropathy, metabolic bone disease and osteoporosis, obesity, and diabetes and pre-diabetic syndromes.

15. A method where the medications in the proceeding claims are supplemented with oral magnesium.

16. A method of treatment where the medications in the proceeding claims are memantine are administered with other therapies such as glycine-site NMDA antagonists, AMPA antagonists, kainate antagonists, calcium channel blockers, partial β and γ secretase inhibitors, anti-oxidants, anti-inflammatory drugs, NOS inhibitors, caspase inhibitors, neurotrophins, or neural stem cell implantation.

17. When the medications in the proceeding claims are used with standard medical therapy.

18. Compositions and method for regulating or limiting malignancies, cancer or tumor growth or proliferation comprising a pharmaceutically effective amount of a compound selected from the group consisting of memantine, felbamate, acamprosate, MRZ 2/579, and mixtures thereof, and methods of administering the same to a patient in need thereof.

19. The compositions and method
